(19) United States
(12) Patent Application Publication
(10) Pub. No.: US 2021/0393968 A1
(43) Pub. Date: Dec. 23, 2021

(54) RADIO FREQUENCY ENERGY HARVESTING

(71) Applicant: Medtronic, Inc., Minneapolis, MN (US)

(72) Inventors: Robert J. Monson, St. Paul, MN (US); Andrew T. Fried, St. Paul, MN (US); Jeffrey P. Bodner, Plymouth, MN (US); Jonathon E. Giftakis, Maple Grove, MN (US); Venkat R. Gaddam, Plymouth, MN (US); Jacob P. Komarek, St. Paul, MN (US)

(21) Appl. No.: 17/350,547

(22) Filed: Jun. 17, 2021

Related U.S. Application Data
(60) Provisional application No. 63/041,622, filed on Jun. 19, 2020.

(51) Int. Cl. A61N 1/378; A61N 1/372; H02J 50/00; H02J 50/20; H02J 7/02; H02J 50/80

(52) U.S. Cl. CPC A61N 1/3787; A61N 1/37223; H02J 50/001; H02J 50/90; H02J 7/02; H02J 50/80; H02J 50/20

(57) ABSTRACT

This disclosure describes devices, systems, and techniques for recharging power sources using RF energy received by one or more antennae. In one example, an implantable medical device includes a rechargeable power supply and an antenna configured to receive radio frequency (RF) energy having one or more frequencies within at least one of a first range from 1 MHz to 20 MHz or a second range from 100 MHz to 700 MHz. The implantable medical device may also include charging circuitry configured to convert the RF energy to a direct current (DC) power and charge the rechargeable power supply with the DC power.

This application claims the benefit of U.S. Provisional Patent Application No. 63/041,622, filed on Jun. 19, 2020 and entitled “RADIO FREQUENCY ENERGY HARVESTING,” the entire contents of which are incorporated herein.

### TECHNICAL FIELD

The disclosure relates to radio frequency energy harvesting, and more particularly, to systems and techniques related to recharging devices using radio frequency energy harvesting.

### BACKGROUND

Electronic devices require energy to operate. For example, a device may include a power source that provides a voltage that drives circuitry of the device. Electronic devices that may include a power source may be implantable medical devices (IMDs). IMDs may be used to monitor a patient condition and/or deliver therapy to the patient. In long term or chronic uses, implantable medical devices may include a rechargeable power source (e.g., comprising one or more capacitors or batteries) that extends the operational life of the medical device to weeks, months, or even years over a non-rechargeable device.

When the energy stored in the rechargeable power source has been depleted, the patient may use an external charging device to recharge the power source. Since the rechargeable power source is implanted in the patient and the charging device is external of the patient, this charging process may be referred to as transcutaneous charging. In some examples, transcutaneous charging may be performed via inductive coupling between a primary coil in the charging device and a secondary coil in the implantable medical device. Therefore, the external charging device can be placed in close proximity to the IMD, but the external charging device does not need to physically connect with the rechargeable power source for charging to occur.

### SUMMARY

This disclosure describes systems, devices, and techniques for recharging power sources via RF energy. A device may operate on electrical power provided by a power supply that can be rechargeable. The device may utilize one or more antennas configured to receive the RF energy such that the device can harvest RF energy from RF signals present in the environment around the device. In some examples, the RF signals may be generated by a variety of sources, such as Wi-Fi devices, cellular telephone towers, Bluetooth devices, or any other source of RF signals. In other examples, the RF signals may be generated by a device configured to transmit RF signals to be received by the one or more antennae (e.g., multiple antennas) of the device with the rechargeable power source. The device receiving the RF energy may be an IMD or any other rechargeable device.

The IMD may include one or more antennae configured to receive RF energy at one or more frequencies. By receiving RF energy at multiple frequencies, the IMD may be configured to harvest more RF energy from various RF signals. In some examples, the RF energy may have a frequency within a range of 1 MHz to 20 MHz and/or 100 MHz to 700 MHz. Signals having these frequencies may transmit through tissue with less absorption when compared to other frequencies. In some examples, the IMD may receive communication information, in addition to charging power, via the received RF energy. An external device may be configured to deliver communications and charging energy via radiated RF signals, either interleaved in time or at different frequencies that the IMD can separate via one or more bandpass filters, for example.

In one example, this disclosure is directed to an implantable medical device that includes a rechargeable power supply, an antenna configured to receive radio frequency (RF) energy having one or more frequencies within at least one of a first range from 1 MHz to 20 MHz or a second range from 100 MHz to 700 MHz, and charging circuitry configured to convert the RF energy to a direct current (DC) power and charge the rechargeable power supply with the DC power.

In another example, this disclosure is directed to a method that includes receiving, via an antenna of an implantable medical device (IMD), radio frequency (RF) energy having one or more frequencies within at least one of a first range from 1 MHz to 20 MHz or a second range from 100 MHz to 700 MHz, converting, by charging circuitry of the IMD, the RF energy to a direct current (DC) power, and charging, by the charging circuitry of the IMD, a rechargeable power supply of the IMD with the DC power.

In another example, this disclosure is directed to a system that includes an external charging device comprising a first antenna configured to radiate radio frequency (RF) energy having one or more frequencies within at least one of a first range from 1 MHz to 20 MHz or a second range from 100 MHz to 700 MHz and an implantable medical device (IMD) that includes a second antenna configured to receive the RF energy and charging circuitry configured to convert the RF energy to a direct current (DC) power and charge the rechargeable power supply with the DC power.

In another example, this disclosure is directed to an implantable medical device that includes a rechargeable power supply, an antenna configured to receive radio frequency (RF) energy, charging circuitry configured to convert a first portion of the RF energy to a direct current (DC) power and charge the rechargeable power supply with the DC power, and communication circuitry configured to convert a second portion of the RF energy to a communication signal and transmit the communication signal to processing circuitry.

In another example, this disclosure is directed to external charging device that includes a directional antenna configured to radiate RF energy, charging circuitry configured to apply an electrical signal to the directional antenna, at least one motor configured to adjust a position of the directional antenna, and processing circuitry configured to receive, via an implantable medical device (IMD), charging information indicative of RF energy received by the IMD and control, based on the charging information, the at least one motor to adjust the position of the directional antenna.

The details of one or more examples are set forth in the accompanying drawings and the description below. Other features, objects, and advantages of examples according to this disclosure will be apparent from the description and drawings, and from the claims.

### BRIEF DESCRIPTION OF DRAWINGS

FIG. 1A is a conceptual diagram illustrating an example of a medical system with multiple stimulation leads implanted along a spinal cord of a patient.

FIG. 1B is a conceptual diagram illustrating an example of a medical system with multiple stimulation leads implanted in the brain of a patient.

FIG. 2A is a block diagram of an example of the implantable medical device of FIG. 1A.

FIG. 2B is a block diagram of an example of the implantable medical device of FIG. 1A.

FIG. 3 is a block diagram of the example of the charging device of FIG. 1A.

FIG. 4 is a block diagram of the example of the external programmer of FIG. 1A.

FIG. 5 is a block diagram of an example directional charging device.

FIG. 6 is a block diagram of an example computer device configured to emit RF signals for charging another device.

FIG. 7 is a flow diagram that illustrates an example technique for charging a power source of a medical device via received RF energy.

FIG. 8 is a flow diagram that illustrates an example technique for adjusting a position for an antenna that transmits RF energy for recharging a medical device.

FIG. 9 is a flow diagram that illustrates an example technique for transmitting RF energy at different frequencies for recharging a medical device.

FIG. 10 is a flow diagram that illustrates an example technique for transmitting RF energy and communication information at different frequencies.

FIG. 11 is a flow diagram that illustrates an example technique for separating charging power from communication information using one or more bandpass filters.

FIG. 12 is a flow diagram that illustrates an example technique for transmitting interleaved RF energy and communication information at the same frequency.

FIG. 13 is a flow diagram that illustrates an example technique for separating interleaved charging power and communication information from received RF energy.

FIG. 14 is a flow diagram that illustrates an example technique for obtaining charging power and communication from transmitted RF energy.

FIG. 15 is a flow diagram that illustrates an example technique for broadcasting RF energy in response to receiving a request to charge.

FIG. 16 is a flow diagram that illustrates an example technique for transmitting a request to transmit RF energy in response to a trigger event.

FIG. 17 is a flow diagram that illustrates an example technique for providing feedback to a user regarding RF energy reception status.

FIG. 18 is a conceptual diagram illustrating an example array of RF energy sources for transmitting RF energy that can be harvested by an implantable medical device.

FIGS. 19A and 19B are conceptual diagrams illustrating example reflectors configured to reflect RF energy to an implantable medical device.

FIG. 20 is a conceptual diagram illustrating an example implantable medical device coupled to a separate antenna.

### DETAILED DESCRIPTION

This disclosure describes systems (e.g., comprising one or more devices, components, sub-systems, or assemblies) and techniques (e.g., methods or processes) for recharging one or more power sources using RF energy harvested from one or more antennae. Many devices utilize power supplies, such as non-rechargeable and rechargeable batteries for operational power. Devices that use non-rechargeable batteries have a limited operation life or require replacement of the non-rechargeable batteries. Rechargeable batteries enable a device to continue operation without replacement of the rechargeable batteries. However, the device must receive power to recharge the batteries, either by attaching a cable to transfer power over a wired connection or by receiving power wirelessly.

Wireless recharging may be completed with an external charger configured to deliver power wirelessly in close contact with the device. For example, an implantable medical device (IMD) may receive wireless power via inductive coupling. However, indicative coupling requires the external coil to be placed in close proximity to the coil of the IMD. Therefore, a charging session may require the patient to hold the external charging device against the skin for the duration of the charging session. Ideally, power would be transferred as fast as possible, but indicative coupling generates heat in the IMD and tissue because not all energy can be transferred to the battery. A limiting factor for charging speed is thus how much heat is generated during charging. Therefore, each charging session can take a considerable amount of time in order to prevent tissue from heating to a point at which damage could occur. If a patient needs to conduct charging sessions once, twice, or more times per day, the duration of these charging sessions can interfere with other patient activities. Even if the patient can remain mobile while charging, the external charging device still must be worn by the patient during the charging session.

As described herein, a device may charge a rechargeable power source using RF energy harvested via one or more antennae. An IMD is described herein as an example device for harvesting RF energy, but any device (e.g., personal computing device, wearable device, smoke detector, clock, or any other electrical device) may harvest RF energy using antennae. In some examples, the antenna may be configured to receive RF signals having one frequency or more than one frequency. The ability to capture energy from multiple frequencies of RF signals may improve the power that the IMD can harvest from various RF signals traveling passed the antenna. Example antennae may include fractal antennas that capture multiple frequencies of RF signals and/or multiple antennas. Since RF signals can be transmitted from large distances, the IMD can receive RF energy and charge a rechargeable power supply without a charging device needed to be disposed in close proximity to the patient or the IMD. In addition, charging the IMD at a distance using RF energy may enable longer charge times and lower charging power such that heat generation during RF charging is no longer an issue for the patient during charging. RF charging in this manner may also facilitate more frequent charging for the patient and reduce the likelihood that the IMD power source is depleted.

For IMDs that are surrounded by tissue, certain frequencies of RF signals may be less attenuated, or less absorbed, by tissue. In this manner, the antenna of the IMD may be configured to capture RF signals having one or more frequencies in at least one of the ranges of 1 to 20 MHz or 100 to 700 MHz. For any charging devices configured to transmit RF energy to IMDs, those charging devices and radiating antenna may be configured to transmit RF energy within those ranges, in some examples. Tissue may, in some examples, change the frequency of RF signals travelling through tissue before those RF signals reach an implanted antenna. In some examples, the IMD may transmit charging information indicative of the power of RF energy received to the charging device, and the charging device may be configured to adjust the frequency of transmitted RF energy in order to achieve frequencies to which the IMD antenna are tuned. This process may improve energy transmission efficiency and reduce heating in tissue. In some examples, a charging device may transmit RF energy at different directions using a directional antenna in order to target the RF energy to the location of the IMD antenna. By using charging information received from the IMD, the charging device may determine which direction targets the IMD antenna and then transmit RF energy at that position to limit RF energy transmission to other portions of the patient at which the IMD is not located. In some embodiments, the charging device may determine the location of the patient without communication with the IMD, for example through the use of one or more cameras (e.g., infrared and/or visual light cameras), microphones, or other devices carried by the patient. The system may additionally or alternatively employ RF triangulation to identify the position of the IMD. The system may, for example, determine the location of the head of the patient by analyzing data from multiple microphones arranged in an array or a circle and direct the antenna according to the location of audible signals from the patient (e.g., breathing sounds or verbalizations from the patient). In other examples, the charging device may receive data from an infrared (IR) camera and determine, from the data from the IR camera, could presence and/or location of the patient and/or the IMD while the patient sleeps or is otherwise in detectable range of the IR camera.

In other examples, the IMD may receive communications from external devices using the RF energy. For example, the same frequency of RF energy may be used to charge the IMD and be a carrier of information. The information may be interleaved with charging power such that the IMD switches between charging the rechargeable power source with the RF energy and determining information from other portions of the received RF energy. The IMD may thus receive information from a charging device using the same antenna. In some examples, different frequencies of the RF energy may be used to provide communications and charging to the IMD. The IMD may include a bandpass filter that passes one or more frequencies of the RF energy to communication circuitry of the IMD. The IMD may pass the remaining frequencies of the RF energy to charging circuitry. In other examples, the IMD may include another bandpass filter that passes different frequencies to the charging circuitry for charging the rechargeable power source. In some examples, the IMD may include multiple antennas to receive RF energy of the same frequency, but one antenna may be configured to pass the RF signals to communication circuitry that derives communication data from the RF energy while another antenna is configured to charge a rechargeable power supply with the RF signals received. In this manner, the transmitting device may transmit such RF signals intended for recharging and communication at higher power than may be necessary for communication alone.

In some examples, the charging devices may be configured for providing RF energy directly to the IMD. In other examples, charging devices may be devices that broadcast RF energy for other purposes, such as Wi-Fi, Bluetooth, cellular telephone communications. The one or more antennae of the IMD may thus be configured to charge a rechargeable power source using RF energy from a specific charging device and/or another device that broadcasts RF signals for other purposes. In some examples, any device may be configured to receive communications from the IMD indicating that RF energy should be transmitted due to low power levels of the rechargeable power source. In some examples, the antenna may be configured according to the proximity to certain RF frequencies. For example, the antenna may be configured to receive frequencies from expected RF energy receivable from various sources (e.g., television broadcasts, Wi-Fi broadcasts, cellular phone tower frequencies, etc.).

While the description of charging (also referred to as “recharging”) an IMD may refer to charging an implantable neurostimulator, the systems and techniques described herein may be used with other types of medical devices or systems. For example, the devices, systems, and techniques described herein may be used with systems including medical devices that deliver electrical stimulation therapy to a patient's heart (e.g., pacemakers, and pacemaker-cardioverter-defibrillators), drug pumps, monitoring devices, or other therapeutic, monitoring, or diagnostic devices.

Although this disclosure generally describes the example of spinal cord stimulation and deep brain stimulation, the systems and techniques described herein may be used to deliver other types of electrical stimulation therapy (e.g., peripheral nerve stimulation, pelvic nerve stimulation, gastric nerve stimulation, or vagal nerve stimulation), stimulation of at least one muscle or muscle groups, stimulation of at least one organ such as gastric system stimulation, stimulation concomitant to gene therapy, and, in general, stimulation of any tissue of a patient. In other examples, non-medical devices may employ the techniques described herein to recharge a power source. Example devices may include wearable computing devices, mobile devices, or any electronic device that benefits from a rechargeable power source.

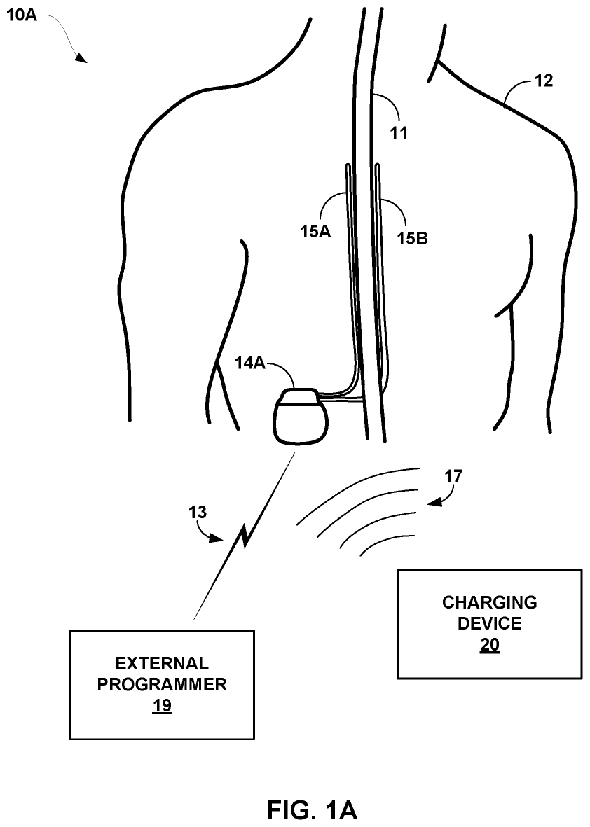

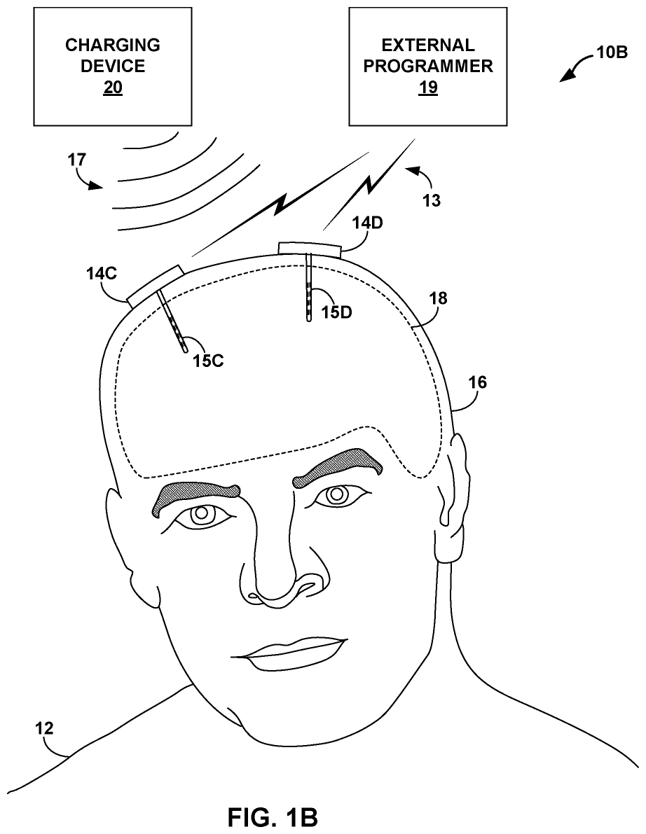

FIG. 1A is a conceptual diagram illustrating an example of a medical system with multiple stimulation leads implanted along spinal cord 11 of patient 12. As shown in the example of FIG. 1A, system 10A includes an implantable medical device (IMD) 110 configured to deliver spinal cord stimulation (SCS) therapy, an external programmer 19, and a charging device 20 in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices. The charging and communication techniques described herein may also be applicable to any medical or non-medical device (e.g., wearable computing device, light, camera, sensor, remote controller, etc.) that charges a rechargeable power source.

As shown in FIG. 1, system 10A includes an IMD 14A, leads 15A and 15B, external programmer 19, and charging device 20, shown in conjunction with a patient 12, who is ordinarily a human patient. In the example of FIG. 1, IMD 14A is an implantable electrical stimulator that is configured to generate and deliver electrical stimulation therapy to patient 12 via one or more electrodes of electrodes of leads 15A and/or 15B (collectively, “leads 15”), e.g., for relief of chronic pain or other symptoms. In other examples, IMD 14A may be coupled to a single lead carrying multiple electrodes or more than two leads each carrying multiple electrodes. IMD 14A may be a chronic electrical stimulator that remains implanted within patient 12 for weeks, months, or even years. In other examples, IMD 14A may be a temporary, or trial, stimulator used to screen or evaluate the efficacy of electrical stimulation for chronic therapy. In one example, IMD 14A is implanted within patient 12, while in another example, IMD 14A is an external device coupled to percutaneously implanted leads. In some examples, IMD 14A uses one or more leads, while in other examples, IMD 14A is leadless.

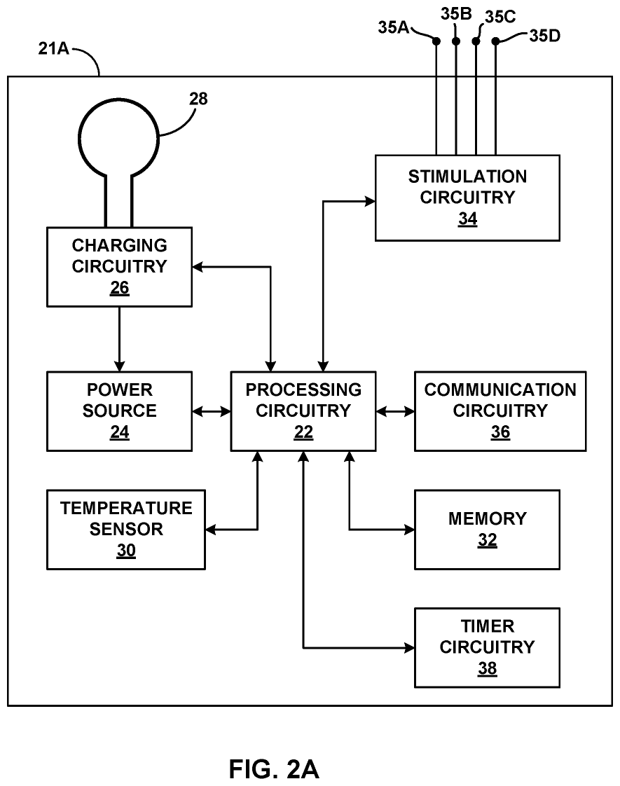

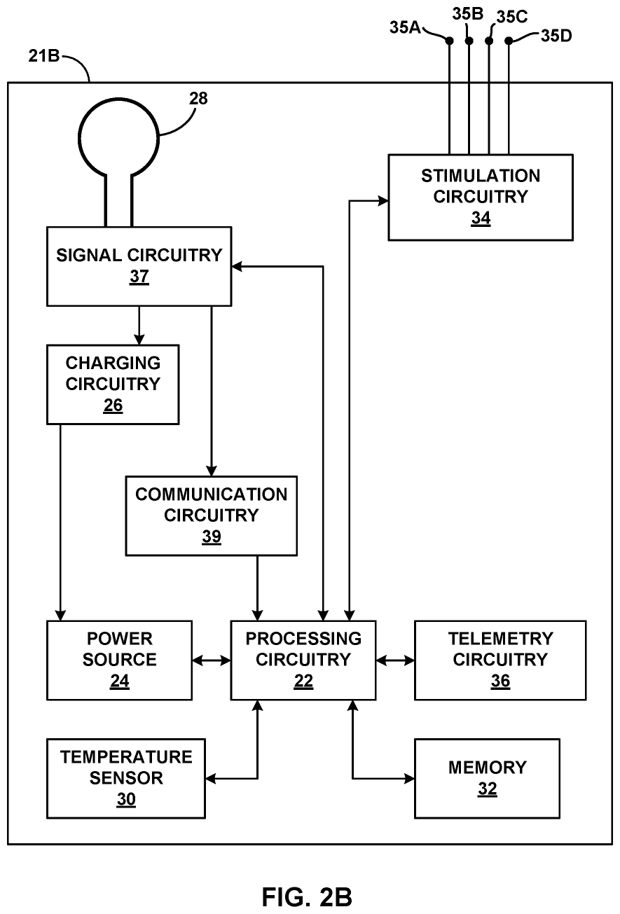

IMD 14A may be constructed of any polymer, metal, or composite material sufficient to house the components of IMD 14A (e.g., components illustrated in FIG. 2) within patient 12. In this example, IMD 14A may be constructed with a biocompatible housing, such as titanium or stainless steel, or a polymeric material such as silicone, polyurethane, ceramic material, or a liquid crystal polymer, and surgically implanted at a site in patient 12 near the pelvis, abdomen, or buttocks. In other examples, IMD 14A may be implanted within other suitable sites within patient 12, which may depend, for example, on the target site within patient 12 for the delivery of electrical stimulation therapy. The outer housing of IMD 14A may be configured to provide a hermetic seal for components, such as a rechargeable or non-rechargeable power source, antennae, etc. In addition, in some examples, the outer housing of IMD 14A is selected from a material that facilitates receiving energy to charge the rechargeable power source.

Electrical stimulation energy, which may be constant current or constant voltage-based pulses, for example, is delivered from IMD 14A to one or more target tissue sites of patient 12 via one or more electrodes (not shown) of implantable leads 15. In the example of FIG. 1, leads 15 carry electrodes that are placed adjacent to the target tissue of spinal cord 11. One or more of the electrodes may be disposed at a distal tip of a lead 130 and/or at other positions at intermediate points along the lead. Leads 15 may be implanted and coupled to IMD 14A. The electrodes may transfer electrical stimulation generated by an electrical stimulation generator in IMD 14A to tissue of patient 12. Although leads 15 may each be a single lead, lead 130 may include a lead extension or other segments that may aid in implantation or positioning of lead 130. In some other examples, IMD 14A may be a leadless stimulator with one or more arrays of electrodes arranged on a housing of the stimulator rather than leads that extend from the housing. In addition, in some other examples, system 10A may include one lead or more than two leads, each coupled to IMD 14A and directed to similar or different target tissue sites.

The electrodes of leads 15 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of leads 15 will be described for purposes of illustration.

The deployment of electrodes via leads 15 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 15 are linear leads having 8 ring electrodes along the axial length of the lead. In another example, the electrodes are segmented rings arranged in a linear fashion along the axial length of the lead and at the periphery of the lead.

The stimulation parameter set of a therapy stimulation program that defines the stimulation pulses of electrical stimulation therapy by IMD 14A through the electrodes of leads 15 may include information identifying which electrodes have been selected for delivery of stimulation according to a stimulation program, the polarities of the selected electrodes, i.e., the electrode combination for the program, voltage or current amplitude, pulse frequency, pulse width, pulse shape of stimulation delivered by the electrodes. These stimulation parameters values that make up the stimulation parameter set that defines pulses may be predetermined parameter values defined by a user and/or automatically determined by system 10A based on one or more factors or user input.

Although FIG. 1 is directed to SCS therapy, e.g., used to treat pain, in other examples system 10A may be configured to treat any other condition that may benefit from electrical stimulation therapy. For example, system 10A may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 10A may be configured to provide therapy taking the form of deep brain stimulation (DBS) as shown in the example of FIG. 1B, peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 12.

In some examples, leads 130 includes one or more sensors configured to allow IMD 14A to monitor one or more parameters of patient 12, such as patient activity, pressure, temperature, or other characteristics. The one or more sensors may be provided in addition to, or in place of, therapy delivery by leads 130.

IMD 14A is configured to deliver electrical stimulation therapy to patient 12 via selected combinations of electrodes carried by one or both of leads 15, alone or in combination with an electrode carried by or defined by an outer housing of IMD 14A. The target tissue for the electrical stimulation therapy may be any tissue affected by electrical stimulation, which may be in the form of electrical stimulation pulses or continuous waveforms. In some examples, the target tissue includes nerves, smooth muscle or skeletal muscle. In the example illustrated by FIG. 1, the target tissue is tissue proximate spinal cord 11, such as within an intrathecal space or epidural space of spinal cord 120, or, in some examples, adjacent nerves that branch off spinal cord 11. Leads 15 may be introduced into spinal cord 11 in via any suitable region, such as the thoracic, cervical or lumbar regions. Stimulation of spinal cord 11 may, for example, prevent pain signals from traveling through spinal cord 11 and to the brain of patient 12. Patient 12 may perceive the interruption of pain signals as a reduction in pain and, therefore, efficacious therapy results. In other examples, stimulation of spinal cord 11 may produce paresthesia which may be reduce the perception of pain by patient 12, and thus, provide efficacious therapy results.

IMD 14A is configured to generate and deliver electrical stimulation therapy to a target stimulation site within patient 12 via the electrodes of leads 15 to patient 12 according to one or more therapy stimulation programs. A therapy stimulation program defines values for one or more parameters (e.g., a parameter set) that define an aspect of the therapy delivered by IMD 14A according to that program. For example, a therapy stimulation program that controls delivery of stimulation by IMD 14A in the form of pulses may define values for voltage or current pulse amplitude, pulse width, pulse rate (e.g., pulse frequency), electrode combination, pulse shape, etc. for stimulation pulses delivered by IMD 14A according to that program.

A user, such as a clinician or patient 12, may interact with a user interface of an external programmer 19 to program IMD 14A. Programming of IMD 14A may refer generally to the generation and transfer of commands, programs, or other information to control the operation of IMD 14A. In this manner, IMD 14A may receive the transferred commands and programs from external programmer 19 to control stimulation, such as electrical stimulation therapy (e.g., informed pulses) and/or control stimulation (e.g., control pulses). For example, external programmer 19 may transmit therapy stimulation programs, stimulation parameter adjustments, therapy stimulation program selections, user input, or other information to control the operation of IMD 14A, e.g., by wireless telemetry or wired connection.

In some cases, external programmer 19 may be characterized as a physician or clinician programmer if it is primarily intended for use by a physician or clinician. In other cases, external programmer 19 may be characterized as a patient programmer if it is primarily intended for use by a patient. A patient programmer may be generally accessible to patient 12 and, in many cases, may be a portable device that may accompany patient 12 throughout the patient's daily routine. For example, a patient programmer may receive input from patient 12 when the patient wishes to terminate or change electrical stimulation therapy, or when a patient perceives stimulation being delivered. In general, a physician or clinician programmer may support selection and generation of programs by a clinician for use by IMD 14A, whereas a patient programmer may support adjustment and selection of such programs by a patient during ordinary use. In other examples, external programmer 19 may include, or be part of, an external charging device that recharges a power source of IMD 14A. In this manner, a user may program and charge IMD 14A using one device, or multiple devices. In some examples, programmer 19 may be a mobile device or cellular phone that is configured to program IMD 14A (e.g., via one or more software applications executed by the phone) and/or charge IMD 14A. In some examples, programmer 19 and a cellular phone or mobile device of patient 12 may be configured to charge IMD 14A.

Information may be transmitted between external programmer 19 and IMD 14A. Therefore, IMD 14A and external programmer 19 may communicate via wireless communication 13 using any techniques known in the art. Examples of communication techniques may include, for example, radiofrequency (RF) telemetry and inductive coupling, but other techniques are also contemplated. In some examples, external programmer 19 includes a communication head that may be placed proximate to the patient's body near the IMD 14A implant site to improve the quality or security of communication between IMD 14A and external programmer 19. Communication between external programmer 19 and IMD 14A may occur during power transmission or separate from power transmission.

Charging device 20 may be configured to provide RF energy 17 to IMD 14A so that IMD 14A can recharge a rechargeable power source using RF energy 17. Charging device 20 may include one or more antennae configured to radiate RF energy having one or more frequencies. Since RF energy may radiate tens or hundreds of feet, charging device 20 may not need to be placed directly on or next to the skin of patient 12. Instead, charging device 20 may be placed somewhere within the room, house, or building at which patient 12 is located. One or more antennae of charging device 20 may be located within or outside of the housing of charging device 20. In some examples, charging device 20 may be configured to move a directional antenna in order to direct RF energy to IMD 14A. Charging device 20 may be a separate device from external programmer 19. In other examples, charging device 20, or the components that provide charging functionality, may be carried by external programmer 19 instead. Charging device 20, or programmer 19 when configured to transmit RF energy, may be configured to transmit RF energy for charging and transmit RF energy for communication in some examples.

In the example of FIG. 1, IMD 14A described as performing a plurality of processing and computing functions. However, external programmer 19 and/or charging device 20 instead may perform one, several, or all of these functions. In this alternative example, IMD 14A functions to relay sensed signals to external programmer 19 for analysis, and external programmer 19 transmits instructions to IMD 14A to adjust the one or more parameters defining the electrical stimulation therapy based on analysis of the sensed signals. One or more devices within system 10A, such as IMD 14A, charging device 20, and/or external programmer 19, may perform various functions as described herein. For example, IMD 14A may include stimulation circuitry configured to deliver electrical stimulation, sensing circuitry, and processing circuitry. However, in other examples, one or more additional devices may be part of the system that performs the functions described herein. For example, IMD 14A may include the stimulation circuitry and the sensing circuitry, but external programmer 19 or other external device may include the processing circuitry that analyzes sensed information.

Although in one example IMD 14A takes the form of an SCS device, in other examples, IMD 14A takes the form of any combination of deep brain stimulation (DBS) devices (e.g., IMDs 14C or 14D of FIG. 1B), implantable cardioverter defibrillators (ICDs), pacemakers, cardiac resynchronization therapy devices (CRT-Ds), left ventricular assist devices (LVADs), implantable sensors, orthopedic devices, or drug pumps, as examples. Moreover, techniques of this disclosure may be used to determine stimulation thresholds (e.g., perception thresholds and detection thresholds) associated any one of the aforementioned IMDs and then use a stimulation threshold to inform the intensity (e.g., stimulation levels) of therapy.

As described herein, IMD 14A may include a rechargeable power supply (not shown) and one or more antennae configured to receive RF energy (e.g., one or more RF signals) having one or more frequencies within at least one of a first range from 1 MHz to 20 MHz or a second range from 100 MHz to 700 MHz. In other examples, the antennae may be configured to receive frequencies in three or more frequency ranges, at least one range of which may be configured to receive frequencies possibly up to 2.4 GHz or even higher. IMD 14A may also include charging circuitry configured to convert the RF energy to a direct current (DC) power and charge the rechargeable power supply with the DC power. In some examples, the RF signal frequency ranges of 1 MHz to 20 MHz and 100 MHz to 700 MHz may include RF signal frequencies that transmit through tissue with less attenuation than frequencies in other ranges. In other examples, the RF signal frequency range may also include 2.4 GHz.

Tissue of patient 12 may be characterized as having a specific absorption rate (SAR) which refers to relative absorption of RF energy as signals pass through tissue. Frequencies with larger SAR values tend to be absorbed at a greater rate than frequencies with lower values. Frequencies with larger SAR values may thus cause greater increase to tissue temperatures and lose more energy that can be received by IMD 14A after the signals have passed through tissue. Therefore, it may be beneficial for IMD 14A to receive RF energy with frequencies that have low SAR values such as within the frequency ranges of 1 MHz to 20 MHz and 100 MHz to 700 MHz. In other examples, at least one appropriate frequency range may be slightly higher, such as 10 MHz to 20 Hz. In some examples, the antenna of IMD 14A may be configured to receive RF energy with frequency in a range of 1 MHz to 1,000 MHz, or even outside of that range. For example, the antenna may be configured to receive RF energy with a frequency from 1 MHz to 20 MHz or even lower frequencies. As another example, the antenna may be configured to receive RF energy higher than 1 GHz, such as 2.4 GHz or 5 GHz which may correspond to RF energy transmitted by other devices for wireless communications. In this manner, IMD 14A may harvest these higher frequencies of RF energy in some examples.

Although SAR values may be higher for some frequencies in this range, the ability to harvest RF energy at these frequencies may contribute to recharge. In one example, the antenna of IMD 14A may be configured to receive RF energy having one or more frequencies within a range from 12 MHz to 16 MHz. In other examples, the antenna of IMD 14A may be configured to receive RF energy having one or more frequencies within a range from 10 MHz to 20 MHz, 10 MHz to 15 MHz, or 1 MHz to 20 MHz. One example frequency for the RF energy may be 13.56 MHz. In some examples, the antenna of IMD 14A is configured to receive RF energy having one or more frequencies within a range from 200 MHz to 500 MHz. In some examples, the antenna of IMD 14A is configured to receive RF energy having one or more frequencies within a range from 250 MHz to 400 MHz. These smaller ranges of frequencies may include frequencies that have lower SAR values than other frequencies outside of the smaller ranges. In one example, the antenna may be configured to receive RF energy having a frequency of approximately 403 MHz. Although an RF signal may include many frequency components, the RF frequencies described herein may be a main frequency at which the RF signals are driven. On a spectral basis, the frequencies of greater power may be those frequencies that fall within the ranges described herein. In some examples, the speed of recharge may be dependent on the magnitude, or power, of the received RF signal. However, if the RF energy is spread across many frequencies, IMD 14A may be configured to charge using energy from many different frequencies as available to be harvested. In some examples, power of certain frequencies may be limited by regulation or to prevent interference with other devices. In this manner, lower power spread over many frequencies may enable for larger overall received power by IMD 14A for charging. As described herein, RF energy charging may be provided in addition to inductive charging using separate antenna such that inductive charging can provide back-up charging capabilities if RF energy harvesting is not sufficient to support IMD 14A energy usage.

In some examples, charging device 20, or two or more charging devices similar to charging device 20, may provide wide band RF energy to facilitate harvesting of RF energy by IMD 14A from different RF frequencies. Wide band RF energy may refer to RF energy broadcast at a plurality of different frequencies. These difference frequencies may be closely packed within a certain frequency band or spread out over larger frequency bands, or over multiple frequency bands, that may correspond to the different frequencies of RF energy that the one or more antennas within IMD 14A may be configured to receive. For example, charging device 20 may broadcast the wide band RF energy using one or more antennas. The wide band RF energy may improve the likelihood that IMD 14A can receive the RF energy at a frequency that may have been shifted slightly due to frequency shift caused by the RF signals passing through the skin of patient 12. Since the frequency of RF signals may be reduced after interacting with tissue, the resulting frequency of the RF energy received by IMD 14A may be lower than the RF signals transmitted by charging device 20. Broadcasting wide band RF energy that covers possible frequency shifts may help to ensure that the one or more antennas of IMD 14A can receive the frequency of the RF energy once it arrives at IMD 14A.

In some examples, the wide band RF energy may have a predetermined frequency band of frequencies at which the RF energy is transmitted. In other examples, charging device 20 may adjust one or more frequencies, or even adjust or shift one or more frequency bands at which RF energy is transmitted. Charging device 20 may operate in an open loop manner and change the frequencies of the RF energy transmitted over time in order to provide the wide band RF transmission. In other examples, charging device 20 may adjust one or more frequencies of the RF energy in response to feedback from a sensing of IMD 14A, another device, or even user feedback on charging via a user interface of external programmer 19 and/or charging device 20.

The frequencies of the wide band RF energy may be selected to be appropriate for use with tissue of patient 12 and/or the surrounding environment. In some examples, charging device 20 may be configured to transmit RF energy in the wide band that does not interfere with communications or electric fields produced by electronics that may be used by patient 12. These other electronics may include other medical devices, automobiles, consumer electronics (e.g., cellular telephones, wireless headphones, laptop computers, microwaves, tools, etc.). In some examples, the wide band RF energy may be configured to include frequencies higher and lower than some of these other devices expected to be near patient 12 during use of charging device 20.

In some examples, the antenna is configured to receive RF energy having a plurality of frequencies, and wherein the charging circuitry is configured to convert the RF energy at the plurality of frequencies to the DC power. For example, a fractal antenna may have multiple legs that enable the antenna to capture RF signals of multiple different frequencies. IMD 14A may include multiple antennae, each of which can generate an electrical signal from the RF signals at one or more frequencies.

In some examples, IMD 14A may also include processing circuitry and communication circuitry that perform different functions. For example, the processing circuitry may be configured to determine a power level of the RF energy received by the antenna and control the communication circuitry to transmit, to charging device 20 that generates the RF energy, an indication of the power level. In this manner, IMD 14A may provide closed-loop feedback regarding the energy received from the RF signals. Charging device 20 may use this indication of power level to adjust an aspect of charging, such as adjusting a transmitted frequency of the RF signal (e.g., to avoid a change in frequency through tissue and/or avoid a frequency that is getting absorbed more by the tissue) or adjusting the location to which the antenna is directed.

IMD 14A may also be configured to receive communication information via RF energy. In one example, charging power and communication information may be interleaved over time in the RF signals. In this manner, IMD 14A may extract communication information from the RF energy by directing the received RF energy to communication circuitry during one period of time and then direct the received RF energy to charging circuitry during another period of time. IMD 14A may determine the interleaving timing by analyzing the RF signals or by receiving separate communication from charging device 20. In this manner, the RF signals intended for charging and the RF signals carrying communication information may have the same (or common) frequency or carrier frequency.

In another example, IMD 14A may separate frequencies of the RF signals associated with communication information from frequencies of the RF signals intended for charging. For example, IMD 14A may include a first bandpass filter configured to pass the first frequency of the first RF energy associated with communication information and a second bandpass filter configured to pass the second frequency of the second RF energy associated with the frequencies for charging the rechargeable power source. In another example, IMD 14A may employ a single bandpass filter to obtain the frequencies containing communication information. IMD 14A may also direct the full spectrum of frequencies of the RF energy received to charging circuitry. In any case, IMD 14A may include processing circuitry configured to determine that the power source is charged to a predetermined threshold and, responsive to determining that the power source is charged to the predetermined threshold, control the charging circuitry to shunt the RF energy received from the antenna.

In some examples, IMD 14A may communicate with charging device 20 regarding one or more aspects of the RF energy received by the one or more antennae. IMD 14A may communicate via any wireless communication technique, such as via an RF transmission protocol (e.g., Bluetooth, Wi-Fi, or other protocol). These aspects may include the received power of the RF energy, detected frequencies, voltage level of the rechargeable power source, or any other aspect. Charging device 20 may adjust one or more parameters that define charging based on the received charging information from IMD 14A. For example, charging device 20 may start or stop transmission of RF energy, change, add, or remove, a frequency of RF signal transmission, or adjust a location to which the one or more directional antennae are directed.

As described herein, charging device 20 may be an external charging device that includes an antenna configured to radiate RF energy having one or more frequencies within at least one of a first range from 1 MHz to 20 MHz or a second range from 100 MHz to 700 MHz. In some examples, the ranges may be slightly different, such as first range from 10 MHz to 20 MHz. Charging device 20 may include or be part of an external program configured to program or otherwise communicate information to IMD 14A. In some examples, charging device 20 may include an antenna that is configured to radiate RF energy at multiple frequencies and/or include multiple antennae configured to radiate RF energy at respective frequencies. As described herein, charging device 20 may include processing circuitry configured to receive an indication of the power level of the RF energy received by IMD 14A and adjust, based on the indication of the power level, the one or more frequencies of the RF energy radiated by the first antenna. Charging device 20 may adjust the transmission frequency because the frequencies of RF signals may change (e.g., decrease) as signals travel through tissue. The magnitude of frequency shift may thus depend on the type of tissue and the thickness of tissue through which the RF signals travel. Therefore, charging device 20 may transmit RF signals with frequencies higher than the RF frequencies IMD 14A is configured to receive to compensate for the frequency shift that can occur from tissue.

In some examples, charging device 20 may include one or more directional antennae configured to radiate RF energy. Charging device 20 may include charging circuitry configured to apply an electrical signal to the directional antenna and at least one motor configured to adjust a position of the directional antenna. For example, the at least one motor may be configured to move the directional antenna about one or more axes in order to direct the RF energy to the IMD 14A position within patient 12. In this manner, the directionality of RF energy may reduce the RF energy absorbed by tissue of patient 12 and reduce unnecessary power use from charging device 20 (e.g., in the case that charging device 20 also operates on a limited power source such as a non-rechargeable or rechargeable battery). Charging device 20 may thus include processing circuitry configured to receive, via IMD 14A, charging information indicative of RF energy received by the IMD and control, based on the charging information, the at least one motor to adjust the position of the directional antenna.

Instead of a directional antenna, charging device 20 may use an electronically steered antenna, such as a beam steering antenna or phased array antenna. These types of antennas may include a multiple-input, multiple-output (MIMO) antenna. Steered antennas may include phased arrays, stacked radiators with different polarizations, and single apertures with multiple feed points. Charging device 20 may adjust the beam of multiple-feed antennas by changing the phase and amplitude of the signals going into the various feeds of the antennas. In some examples, phased-array antennas operate by creating phase and/or amplitude shifting in the radio frequency (RF) path to steer beams in a particular direction. In other examples, beam steering circuitry may use a local oscillator phase-shifting approach.

In some examples, the processing circuitry of charging device 20 may be configured to control the at least one actuator (e.g., a motor or other mechanical movement device) to sweep the directional antenna through a plurality of positions, control the charging circuitry to apply the electrical signal to the directional antenna at each position of the plurality of positions, receive, via IMD 14 A, charging information indicative of the RF energy received by the IMD at each position of the plurality of positions, and control the at least one motor to adjust the position of the directional antenna by selecting one position of the plurality of positions for subsequent radiation of RF energy by the directional antenna. In this manner, charging device 20 may be configured to sweep through a plurality of directions for the directional antenna in order to find the appropriate direction at which IMD 14A is located. Instead of a full sweep, charging device 20 may also be able to monitor and adjust the direction of RF energy transmission during the charging session. For example, charging device 20 may be configured to detect, based on the charging information, a reduction in power of the RF energy received by the IMD. This reduction in power may be due to patient 12 moving with respect to the location of charging device 20. Then, charging device can, responsive to detect the reduction in power, control the at last one motor to adjust the position of the directional antenna.

In some examples, charging device 20 may not be constructed for the sole purpose of charging IMD 14A. Instead, charging device 20 may be a computing device (e.g., a cellular phone, mobile phone, or smart device) that includes an antenna configured to radiate RF energy receivable by IMD 14A. For example, a mobile phone may have an antenna configured to receive data in certain frequencies for 3G or 4G communication (e.g., frequencies that may be within a range of around 700 MHz to 900 MHz or higher frequency bands). The mobile phone may include charging circuitry that can instead apply a signal to the antenna in order to radiate RF energy from the antenna for harvesting by IMD 14A. In some examples, charging device 20 may separately broadcast RF energy independently from the operation of IMD 14A. For example, charging device 20 may control a RF transmitter (e.g., circuitry and antenna) to broadcast RF energy continuously or as the function of the computer device allows. For example, a cellular phone may broadcast RF energy in response to receiving a trigger signal. The cellular phone may generate the trigger signal in response to determining a particular time of day (e.g., nighttime indicative of patient 12 sleeping), the cellular phone battery has a charge exceeding a predetermined threshold, the RF antenna is not currently used for other functions of the cellular phone (e.g., no other transmission and/or receiving of RF signals for communication), or the cellular phone has available computing and/or antenna bandwidth to transmit RF signals.

In some examples, the computing device, such as the cellular phone, may receive communications directly from IMD 14A or a user related to the function of IMD 14A. For example, the cellular phone can receive a user input or signal from IMD 14A that IMD 14A has a sub-threshold battery voltage and requires recharging. The cellular phone may thus begin broadcasting RF energy in an attempt to charge IMD 14A. Other purpose built charging devices may be portable and sized to be carried within a pocket, for example, of the patient such that the charging device can transmit RF energy to the IMD 14A or other medical device carried on the patient.

FIG. 1B is a conceptual diagram illustrating an example of a medical system 10B with multiple stimulation leads 15D implanted in brain 18 of patient 12. In the example of FIG. 1, medical system 10B includes charging device 20 configured to deliver energy to one or more implantable medical devices (IMDs) 14C and 14D such as via RF energy 17. For ease of description, IMDs 14C and 14D may be collectively referred to as “IMDs 14.” In an example, IMDs 14 may be at least partially or fully implanted within patient 12. IMDs 14 may include or be coupled to a respective lead (e.g., lead 15C coupled to IMD 14C, and lead 15D coupled to IMD 14D). One or more electrodes of lead 15C and lead 15D are configured to provide electrical signals (e.g., pulses or analog signals) to surrounding anatomical regions of brain 18 in a therapy that may alleviate a condition of patient 12. In some examples, one or both of IMDs 14 may be coupled to more than one lead implanted within brain 18 of patient 12 to stimulate multiple anatomical regions of the brain. In an example, such as shown in FIG. 1, system 10 may include two IMDs 14 that each include a lead. However, more than two IMDs may be disposed in patient 12 in other examples. External programmer 19 may be configure send and/or receive information from IMDs 14 via communication signals 13. IMDs 14C and 14D may be configured to include similar components and provide similar functionality as described with respect to IMD 14A of FIG. 1A.

Deep brain stimulation (DBS) delivered by one or both of IMDs 14 may treat dysfunctional neuronal activity in the brain which manifests as diseases or disorders such as Huntington's Disease, Parkinson's Disease, or movement disorders. Certain anatomical regions of brain 18 may be responsible for producing the symptoms of such brain disorders. As one example, stimulating an anatomical region, such as the Substantia Nigra, in brain 18 may reduce the number and/or magnitude of tremors experienced by patient 12. Other anatomical regions that may receive stimulation therapy include the subthalamic nucleus, globus pallidus interna, ventral intermediate, and zona inserta. Anatomical regions such as these are targeted by the clinician during pre-operative planning and lead implantation. In other words, the clinician may attempt to position the leads 15C and 15D as close to these regions as possible for DBS therapy.

Typical DBS leads include one or more electrodes placed along the longitudinal axis of the lead, such may be seen on leads 15C and 15D. In one example, each electrode may be a ring electrode that resides along the entire circumference of the lead at one axial location on the lead. Therefore, electrical current from the ring electrodes propagates in all directions from the active electrode. The resulting stimulation field reaches anatomical regions of brain 18 within a certain distance of the lead in all directions. In other examples, lead 15C or 15D may have a complex electrode array geometry. A complex electrode array geometry include a plurality of electrodes positioned at different axial positions along the longitudinal axis of the lead and a plurality of electrodes positioned at different angular positions around the circumference of the lead (which may be referred to as electrode segments). In some examples, this disclosure may be applicable to leads having all ring electrodes, or one or more ring electrodes in combination with electrode segments at different axial positions and angular positions around the circumference of the lead. In this manner, electrodes may be selected along the longitudinal axis of leads 15C and 15D and along the circumference of the lead. A complex electrode array geometry may allow activating a subset of electrodes of leads 15C and 15D selected to produce customizable stimulation fields that may be directed to a particular side of lead 15C or 15D in order to isolate the stimulation field around the target anatomical region of brain 18. IMDs 14 may be implanted on cranium 16, such as shown in FIG. 1B. IMDs 14 may be positioned elsewhere on cranium 16, such as closer together or further apart than shown in FIG. 1B.

FIG. 2A is a block diagram of an example of the implantable medical device 21A. IMD 21A may be an example of any of IMDs 14A, 14C, 14D, or another medical device. In the example of FIG. 2A, IMD 21A includes processing circuitry 22, power source 24 (e.g., a rechargeable power source), charging circuitry 26, coil 28 (also may be referred to as secondary coil 28), temperature sensor 30, memory 32, stimulation circuitry 34, communication circuitry 36, and timer circuitry 38. In other examples, IMD 21A may include a greater or fewer number of components.

In general, IMD 21A may include any suitable arrangement of hardware, alone or in combination with software and/or firmware, to perform the various techniques described herein attributed to IMD 21A or processing circuitry 22. In various examples, IMD 21A may include one or more processors (e.g., processing circuitry 22), such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 21A also, in various examples, may include a memory 32, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors (e.g., processing circuitry) to perform the actions attributed to them. Moreover, although processing circuitry 22, stimulation circuitry 34, charging circuitry 26, and communication circuitry 36 are described as separate, in some examples, processing circuitry 22, stimulation circuitry 34, charging circuitry 26, and communication circuitry 36 are physically and/or functionally integrated. In some examples, processing circuitry 22, stimulation circuitry 34, charging circuitry 26, and communication circuitry 36 correspond to individual hardware units, such as ASICs, DSPs, FPGAs, or other hardware units.

Memory 32 may be configured to store therapy programs or other instructions that specify therapy parameter values for the therapy deliverable by stimulation circuitry 34 and IMD 21A. In some examples, memory 32 may also store temperature data from temperature sensor 30, temperature thresholds, instructions for recharging power source 24, circuit models, open-circuit voltage models, tissue models, thresholds, instructions for communication between IMD 21A and programmer 19 or charging device 20, or any other instructions required to perform tasks attributed to IMD 21A. In this manner, memory 32 may be configured to store charge states of one or more rechargeable power sources.

Generally, stimulation circuitry 34 may be configured to generate and deliver electrical stimulation under the control of processing circuitry 22. In some examples, processing circuitry 22 controls stimulation circuitry 34 by accessing memory 32 to selectively access and load at least one of the stimulation programs to stimulation circuitry 34. For example, in operation, processing circuitry 22 may access memory 32 to load one of the stimulation programs to stimulation circuitry 34. In such examples, relevant stimulation parameters may include a voltage amplitude, a current amplitude, a pulse rate, a pulse width, a duty cycle, or the combination of electrodes 35A, 35B, 35C, and 35D (or fewer or greater electrodes) that stimulation circuitry 34 uses to deliver the electrical stimulation signal. Although stimulation circuitry 34 may be configured to generate and deliver electrical stimulation therapy via one or more of electrodes 35A, 35B, 35C, and 35D of a lead (e.g., leads 15A, 15C, or 15D), stimulation circuitry 34 may be configured to provide different therapy to patient 12. For example, stimulation circuitry 34 may be configured to deliver drug delivery therapy via a catheter. These and other therapies may be provided by IMD 21A.

IMD 21A also includes components configured to receive power from charging device 20 to recharge power source 24, such as when power source 24 has been at least partially depleted. As shown in FIG. 2A, IMD 21A includes antenna 28 and charging circuitry 26 coupled to power source 24. Charging circuitry 26 may be configured to charge power source 24 with power received from external charging device 20 or any other RF energy in the environment around IMD 21A. The power generated by external charging device 20 is, in some examples, generated according to a selected power level determined by either processing circuitry 22 or charging device 20. Although processing circuitry 22 may provide some commands to charging circuitry 26 in some examples, processing circuitry 22 may not need to control any aspect of recharging in other examples. IMD 21A may direct power from antenna 28 directly to one or more components to enable operation of IMD 21A. In other examples, IMD 21A may include no power source 24 and instead direct all energy received from antenna 28 to the operation of IMD 21A.

In other examples, IMD 21A may include, instead of or in addition to a power source 24, a primary cell battery. For example, IMD 21A may primarily draw power from a rechargeable battery of power source 24. In response to detecting that the remaining voltage of rechargeable power source is no longer capable of operating IMD 21A, or can no longer operate certain functions, processing circuitry 22 or circuitry of power source 24 may switch to draw power from the primary battery that is non-rechargeable. In this manner, the primary battery may operate to provide backup or reserve capacity in a situation where the rechargeable battery has not been recharged. In some examples, the primary cell battery may have sufficient charge to operate for weeks, months, or even one year or longer. Therefore, the primary cell battery may support operation even if the patient is unable to charge for extended periods of time due to travel, charger unavailability, or any other issue. If the primary cell battery is depleted, IMD 21A may still operate solely on the rechargeable battery that relies on charging sessions. IMD 21A may communicate the charge status of the primary cell batter to an external programmer or other device along with normal operational data. In some examples, IMD 21A may transmit a low charge or no charge notification to the programmer or other device in response to detecting that the primary cell battery has been depleted.

Antenna 28 may include a coil of wire or other device in which an electrical current can be induced via interaction with the RF signals. Although antenna 28 is illustrated as a simple loop in FIG. 2A, antenna 28 may include multiple turns of wire, one or more straight legs, a fractal antenna design, or any other configuration that may or may not be tuned to specific RF signal frequencies. The induced electrical current may then be used by charging circuitry 26 of IMD 21A to recharge power source 24. Any of these techniques may generate heat in IMD 21A that may be monitored, for example, by temperature sensor 30.

In some examples, IMD 21A may include multiple antennas configured to receive energy of different frequencies and/or for different purposes (e.g., charging and/or communication). For example, IMD 21A may include at least one antenna configured to harvest RF energy, at least one antenna configured to receive direct battery charging (e.g., inductive coupling via an inductive coil), and at least one antenna that supports receiving and/or transmitting communication data with another device (e.g., an implanted medical device or an external programmer or other device). In the case of multiple antennas configured to receive energy to charge power source 24, IMD 21A may charge one or more rechargeable batteries of power source 24 with the current obtained via all of the multiple antennas. Depending on the available capacity of the rechargeable power source, IMD 21A may selectively choose which antenna is used to recharge the power supply.

The different antennas may support charging using different modalities simultaneously or interleaved over time. Although RF energy is generally described herein, IMD 21A may, for example, additionally include a coil for inductive coupling for charging or communication purposes. In this example, the inductive coupling coil may receive energy directly from a external inductive coupling coil placed near the skin of the patient. However, IMD 21A may also harvest energy from RF signals received from a different antenna (e.g., antenna 28). In some examples, IMD 21A may switch between the different antennas and respective energy transfer modalities as needed. For example, during inductive coupling, IMD 21A may disconnect the RF energy harvesting antenna because inductive coupling may interfere with the reception of the RF signals. In some examples, IMD 21A may periodically request that the charging device pauses inductive coupling, or the charging device may independently pause inductive coupling, to enable IMD 21A to receive communications from other devices that may not be detectable during the power transfer during inductive coupling.

Charging circuitry 26 may include one or more circuits that filter and/or transform the electrical signal induced in antenna 28 to an electrical signal capable of recharging power source 24. For example, charging circuitry 26 may include a half-wave rectifier circuit and/or a full-wave rectifier circuit configured to convert alternating current from the RF energy to a direct current for power source 24. A full-wave rectifier circuit may be more efficient at converting the RF energy for power source 24. However, a half-wave rectifier circuit may be used to store energy in power source 24 at a slower rate. In some examples, charging circuitry 26 may include both a full-wave rectifier circuit and a half-wave rectifier circuit such that charging circuitry 26 may switch between each circuit to control the charging rate of power source 24 and temperature of IMD 21A.

In some examples, charging circuitry 26 may include a tank circuit, which may include antenna 28. The tank circuit may be tuned to the external antenna in order to generate electrical current that charges power source 24. However, in some cases, IMD 21A may include circuitry that is configured to change the resonant frequency of the tank circuit, or tune the tank circuit, as desired. The resonant frequency of the tank circuit may be changed by variable reactance provided by a variable capacitance. For example, IMD 21A may include a tuning switch that receives a control signal from processing circuitry 22 to alter the state and ultimately vary the reactance of the tank circuit that includes antenna 28. The tuning switch may open and close to remove or add a capacitor in parallel with a hardwired capacitor, where the hardwired capacitor is in series with antenna 28. In this manner the tuning switch may tune the tank circuit for recharge or tune the tank circuit to a resonant frequency other than the recharge frequency to provide power management by reducing the received power during recharge (e.g., detune the tank circuit). Other types of circuitry may also be used by charging circuitry 26 in order to detune antenna 28 and change the electrical current generated by antenna 28 from the power output by the external antenna.

In some examples, charging circuitry 26 may include a measurement circuit (e.g., a coulomb counter) configured to measure the current and/or voltage induced in IMD 21A during inductive coupling. This measurement may be used to measure or calculate the power transmitted to power source 24 of IMD 21A from charging device 20. In some examples, charging circuitry 26 or other circuitry may include an electrometer or kilometer, which may measure the charge current being applied to power source 24 and communicate this charge current to processing circuitry 22. In some examples, processing circuitry 22 may control charging circuitry 26 to open a circuit of charging circuitry 26 to prevent electrical induction and/or detune antenna 28 of IMD 21A to generate less power from charging device 20. In other examples, charging circuitry may include a shunt that can operate to shunt unneeded power from power source 24.

Power source 24 may include one or more capacitors, batteries, and/or other energy storage devices. Power source 24 may then deliver operating power to the components of IMD 21A. In some examples, power source 24 may include a power generation circuit to produce the operating power. Power source 24 may be configured to operate through hundreds or thousands of discharge and recharge cycles. Power source 24 may also be configured to provide operational power to IMD 21A during the recharge process. In some examples, power source 24 may be constructed with materials to reduce the amount of heat generated during charging. In other examples, IMD 21A may be constructed of materials that may help dissipate generated heat at power source 24, charging circuitry 26, antenna 28 over a larger surface area of the housing of IMD 21A.

Although power source 24, charging circuitry 26, and antenna 28 are shown as contained within the housing of IMD 21A, at least one of these components may be disposed outside of the housing. For example, secondary coil 28 may be disposed outside of the housing of IMD 21A to facilitate better coupling between secondary coil 28 and the primary coil of charging device 20. These different configurations of IMD 21A components may allow IMD 21A to be implanted in different anatomical spaces or facilitate better reception of RF energy.

IMD 21A may also include temperature sensor 30. Temperature sensor 30 may include one or more temperature sensors (e.g., thermocouples or thermistors) configured to measure the temperature of IMD 21A. Temperature sensor 30 may be disposed internal of the housing of IMD 21A, contacting the housing, formed as a part of the housing, or disposed external of the housing. Temperature sensor 30 positioned within the IMD and may sense an internal temperature of the IMD. In an example, temperature sensor 30 may sense a temperature of the housing of the IMD. In other examples, temperature sensor 30 may be positioned on the housing of the IMD and it may sense the temperature of the tissue surrounding the IMD. Multiple temperature sensors may be positioned on or within the IMD in some examples.

As described herein, temperature sensor 30 may be used to directly measure the temperature of IMD 21A and/or tissue surrounding and/or contacting the housing of IMD 21A. Processing circuitry 22, or charging device 20, may use this temperature measurement as tissue temperature to determine a temperature model of IMD 21A or of the tissue surrounding IMD 21A. Although a single temperature sensor may be adequate, multiple temperature sensors may provide a better temperature gradient or average temperature of IMD 21A. The various temperatures of IMD 21A may also be modeled. Although processing circuitry 22 may continually measure temperature using temperature sensor 30, processing circuitry 22 may conserve energy by only measuring temperature during recharge sessions. Further, temperature may be sampled at a rate to determine adequate temperature measurements or models, but the sampling rate may be reduced to conserve power as appropriate.

Processing circuitry 22 may also control the exchange of information with charging device 20 and/or an external programmer using communication circuitry 36. Communication circuitry 36 may be configured for wireless communication using radio frequency protocols or inductive communication protocols. Communication circuitry 36 may include one or more antennas configured to communicate with charging device 20, for example. Processing circuitry 22 may transmit operational information and receive therapy programs or therapy parameter adjustments via communication circuitry 36. Also, in some examples, IMD 21A may communicate with other implanted devices, such as stimulators, control devices, or sensors, via communication circuitry 36. In addition, communication circuitry 36 may be configured to transmit the measured tissue temperatures from temperature sensor 30, the charge state of power source 24, for example. In some examples, tissue temperature may be measured adjacent to power source 24.

In other examples, processing circuitry 22 may transmit additional information to charging device 20 related to the operation of power source 24. For example, processing circuitry 22 may use communication circuitry 36 to transmit indications that power source 24 is completely charged, power source 24 is fully discharged, how much charge (e.g., the charge current) is being applied to power source 24, the charge capacity of power source 24, the state-of-charge (SOC) of power source 24, or any other charge information of power source 24. Processing circuitry 22 may also transmit information to charging device 20 that indicates any problems or errors with power source 24 that may prevent power source 24 from providing operational power to the components of IMD 21A.

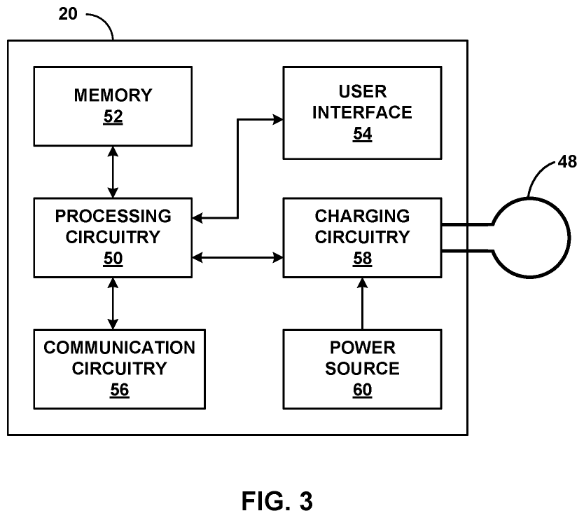

Processing circuitry 22 may determine the charge state of power source 24. For example, processing circuitry 22 may include a voltage tester circuit coupled to power source 24 to determine the charge state (e.g., voltage level) of power source 24. In some examples, processing circuitry 22 determines the charge state as a voltage measurement value, as a percentage of full capacity, in relation to another power source charge state (e.g., higher, same, similar, lower), or any combination thereof. In some examples, a user interface (e.g., user interface 54 of FIG. 3) indicates the charge state of one or more power sources. For example, the user interface may display a bar chart, graph, value, a light, or any other indication of charge state of the power source.

In an example, processing circuitry 22 may control timer circuitry 38 to begin a countdown, such as during a recharge session. In an example, processing circuitry 22 may control one or more devices to perform a particular task with a particular duration, such as may be timed via timer circuitry 38. For example, processing circuitry 22 may control charging circuitry 26 to open a circuit for a desired amount of time (e.g., on the scale of seconds, minutes, or hours). Once the countdown expires, processing circuitry 22 may control charging circuitry 26 to close the circuit, such as to tune the IMD to the charging device (e.g., change from a detuned state of the IMD).

FIG. 2B is a block diagram of an example of IMD 14A, 14C, and 14D of FIGS. 1A and 1B. As shown in FIG. 2B, IMD 21B is substantially similar to IMD 21A of FIG. 2A. However, IMD 21B can derive communication information from RF signals in addition to harvesting energy from the RF signals. For example, antenna 28 is coupled to signal circuitry 37. Signal circuitry 37 can determine which portions of the RF signals are sent to charging circuitry 26 or communication circuitry 39. In one example, signal circuitry 37 may be configured to send the full spectrum of RF energy to each of charging circuitry 26 and communication circuitry 39. Then, each of charging circuitry 26 and/or communication circuitry 39 may include respective filters (e.g., low pass, high pass, or bandpass filters) or other signal processing or signal conditioning circuitry.

In other examples, signal circuitry 37 may include one or more filters or switches that control when signal circuitry 37 sends some or all of the RF energy to one or both of charging circuitry 26 or communication circuitry 39. In some examples, processing circuitry 22 may directly control the functionality of signal circuitry 37, such identifying when the RF signals include communication information. In other examples, signal circuitry 37 may include an ASIC or other processing circuitry to independently control RF signal transmission to other components within IMD 21B. In one example, signal circuitry 37 may include a bandpass filter to only send a predetermined frequency range to communication circuitry 39 associated with communication sent from charging device 20. Signal circuitry 37 may transmit a full spectrum of the RF signal then to charging circuitry 26. In another example, signal circuitry 37 may include a first bandpass filter to send a first predetermined frequency range to communication circuitry 39 associated with communication sent from charging device 20 and a second bandpass filter to send a second predetermined frequency range to charging circuitry 26. In this situations, the communication information may be using a different frequency than the frequency of the RF signals used for charging.

In other examples, the same RF signal frequency may be used for charging and communication. Therefore, signal circuitry 37 may separate the RF signals on a time interleaved basis because the communication information and the charging energy may be time interleaved from charging device 20. Signal circuitry 37 may detect a flag in the RF signals indicating when different portions of the interleaved signal arrives or operate on a predetermined timing pattern. In any case, signal circuitry 37 may function to derive both communications and recharge power from received RF signals via antenna 28.

FIG. 3 is a block diagram of the example of charging device 20. While charging device 20 may generally be described as a hand-held device, charging device 20 may be a larger portable device or a more stationary device in other examples. In addition, in other examples, charging device 20 may be included as part of an external programmer (e.g., programmer 19 shown in FIGS. 1A and 1B) or include functionality of an external programmer. Charging device 20 may be configured to communicate with an external programmer, external server via a network, or other computing device. As illustrated in FIG. 3, charging device 20 may include antenna 48, processing circuitry 50, memory 52, user interface 54, communication circuitry 56, charging circuitry 58, and power source 60. Memory 52 may store instructions that, when executed by processing circuitry 50, cause processing circuitry 50 and external charging device 20 to provide the functionality ascribed to external charging device 20 throughout this disclosure.

In general, charging device 20 includes any suitable arrangement of hardware, alone or in combination with software and/or firmware, to perform the techniques attributed to charging device 20, and processing circuitry 50, user interface 54, communication circuitry 56, and charging circuitry 58 of charging device 20. In various examples, charging device 20 may include one or more processors (e.g., processing circuitry 50), such as one or more microprocessors, DSPs, ASICs, FPGAs, or any other equivalent integrated or discrete logic circuitry, as well as any combinations of such components. Charging device 20 also, in various examples, may include a memory 52, such as RAM, ROM, PROM, EPROM, EEPROM, flash memory, a hard disk, a CD-ROM, comprising executable instructions for causing the one or more processors to perform the actions attributed to them. Moreover, although processing circuitry 50 and communication circuitry 56 are described as separate, in some examples, processing circuitry 50 and communication circuitry 56 are functionally integrated. In some examples, processing circuitry 50 and communication circuitry 56 and charging circuitry 58 correspond to individual hardware units, such as ASICs, DSPs, FPGAs, or other hardware units.

Memory 52 may store instructions that, when executed by processing circuitry 50, cause processing circuitry 50 and charging device 20 to provide the functionality ascribed to charging device 20 throughout this disclosure. For example, memory 52 may include instructions that cause processing circuitry 50 to control charging circuitry 58, communicate with IMD 14, or instructions for any other functionality. In addition, memory 52 may include a record of selected power levels, calculated estimated energy transfers, or any other data related to charging rechargeable power source 24. Processing circuitry 50 may, when requested, transmit any of this stored data in memory 52 to another computing device for review or further processing.

In some examples, memory 52 may be configured to store measured charge states of one or more power sources of one or more IMDs over time, age of a power source 24, and/or any other factors that may affect voltage of a power source 24. In some examples, memory 52 may be configured to store data representative of an energy absorption tissue model used by processing circuitry 50 to determine the energy absorption of tissue at a particular operating frequency. In some examples, memory 52 may be configured to store data representative of a tissue model used by processing circuitry 50 to calculate tissue temperature based on tissue model and power transmitted to rechargeable power source 24 over a period of time. Tissue model may indicate how temperate of tissue surrounding IMD 14 changes over time.

User interface 54 may include a button or keypad, lights, a speaker that generates audible sounds, a microphone that detects voice commands, a display, such as a liquid crystal (LCD), light-emitting diode (LED), or cathode ray tube (CRT). In some examples the display may be a touch screen. As discussed in this disclosure, processing circuitry 50 may present and receive information relating to the charging of rechargeable power source 24 via user interface 54. For example, user interface 54 may indicate when charging is occurring, quality of the alignment between secondary coil 28 and primary coil 48, the selected power level, current charge level of rechargeable power source 24, duration of the current recharge session, anticipated remaining time of the charging session, or any other information. Processing circuitry 50 may receive some of the information displayed on user interface 54 from IMD 14 in some examples.

User interface 54 may also receive user input via user interface 54. The input may be, for example, in the form of pressing a button on a keypad or selecting an icon from a touch screen. The input may request starting or stopping a recharge session, a desired level of charging, or one or more statistics related to charging rechargeable power source 24 (e.g., the estimated energy transfer). In this manner, user interface 54 may allow the user to view information related to the charging of rechargeable power source 24 and/or receive charging commands.

Charging device 20 also includes components to transmit power to recharge rechargeable power source 24 associated with IMD 14. As shown in FIG. 3, charging device 20 includes antenna 48 and charging circuitry 58 coupled to power source 60. Charging circuitry 58 may be configured to apply an electrical signal to antenna 48 that causes antenna 48 to radiate RF signals in one or more frequencies. Although antenna 48 is illustrated as a simple loop in the example of FIG. 3, antenna 48 may include multiple turns of wire, one or more straight legs, one or more geometric shapes, or any other shape configured to radiate at any frequency described herein. In one example, antenna 48 may be a fractal antenna configured to radiate one or more frequencies of RF signals. In addition, charging device 20 may include two or more antennas in other examples. Charging circuitry 58 may generate the electrical current according to a power level selected by processing circuitry 50 based on the estimated energy transfer.

Charging circuitry 58 may include one or more circuits that generate an electrical signal that is transmitted to antenna 48. Charging circuitry 58 may generate an alternating current of specified amplitude and frequency in some examples. In other examples, charging circuitry 58 may generate a direct current. In any case, charging circuitry 58 may be configured to generate electrical signals that, in turn, causes antenna 48 to radiate RF signals that can be captured by an IMD or other device. In this manner, charging circuitry 58 may be configured to charge rechargeable power source 24 of IMD 21A, for example.

Power source 60 may deliver operating power to the components of charging device 20. Power source 60 may also deliver the operating power to drive antenna 48 during the charging process. Power source 60 may include a battery and a power generation circuit to produce the operating power. In some examples, the battery may be rechargeable to allow extended portable operation. In other examples, power source 60 may draw power from a wired voltage source such as a consumer or commercial power outlet.

Although power source 60 and charging circuitry 58 are shown within a housing of charging device 20, and antenna 48 is shown external to charging device 20, different configurations may also be used. For example, antenna 48 may also be disposed within the housing of charging device 20. In another example, power source 60, charging circuitry 58, and antenna 48 may be all located external to the housing of charging device 20 and coupled to charging device 20.

Communication circuitry 56 supports wireless communication between IMD 21A, charging device 20, and/or programmer 19 under the control of processing circuitry 50. Communication circuitry 56 may also be configured to communicate with another computing device via wireless communication techniques, or direct communication through a wired connection. In some examples, communication circuitry 56 may be substantially similar to communication circuitry 36 of IMD 21A described herein, providing wireless communication via an RF or proximal inductive medium. In some examples, communication circuitry 56 may include an antenna, which may take on a variety of forms, such as an internal or external antenna. In some examples, communication to IMD 21A may take place via modulation of power from antenna 48 that is detectable by IMD 21A.

Examples of local wireless communication techniques that may be employed to facilitate communication between charging device 20 and IMD 21A include RF communication according to the 802.11 or Bluetooth specification sets or other standard or proprietary telemetry protocols. In this manner, other external devices may be capable of communicating with charging device 20 without needing to establish a secure wireless connection.

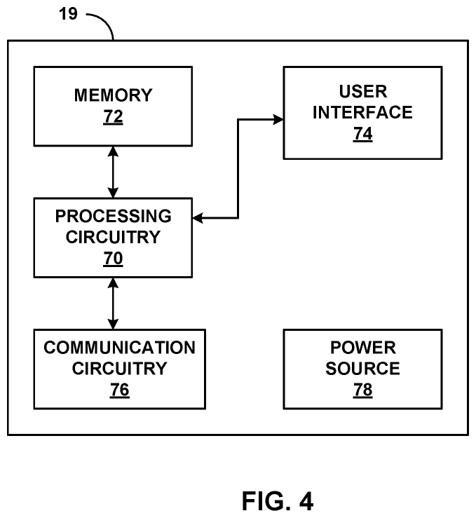

FIG. 4 is a block diagram of the example of external programmer 19 of FIGS. 1A and 1B. Although programmer 19 may generally be described as a hand-held device, programmer 19 may be a larger portable device or a more stationary device. In some examples, programmer 19 may be referred to as a tablet computing device. In addition, in other examples, programmer 19 may be included as part of an external charging device or include the functionality of an external charging device. As illustrated in FIG. 3, programmer 19 may include a processing circuitry 70, memory 72, user interface 74, communication circuitry 76, and power source 78. Memory 72 may store instructions that, when executed by processing circuitry 70, cause processing circuitry 70 and external programmer 19 to provide the functionality ascribed to external programmer 19 throughout this disclosure. Each of these components, or modules, may include electrical circuitry that is configured to perform some or all of the functionality described herein. For example, processing circuitry 70 may include processing circuitry configured to perform the processes discussed with respect to processing circuitry 70.

In general, programmer 19 comprises any suitable arrangement of hardware, alone or in combination with software and/or firmware, to perform the techniques attributed to programmer 19, and processing circuitry 70, user interface 74, and communication circuitry 76 of programmer 19. In various examples, programmer 19 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. Programmer 19 also, in various examples, may include a memory 72, such as RAM, ROM, PROM, EPROM, EEPROM, flash memory, a hard disk, a CD-ROM, comprising executable instructions for causing the one or more processors to perform the actions attributed to them. Moreover, although processing circuitry 70 and communication circuitry 76 are described as separate modules, in some examples, processing circuitry 70 and communication circuitry 76 may be functionally integrated with one another. In some examples, processing circuitry 70 and communication circuitry 76 correspond to individual hardware units, such as ASICs, DSPs, FPGAs, or other hardware units.

Memory 72 (e.g., a storage device) may store instructions that, when executed by processing circuitry 70, cause processing circuitry 70 and programmer 19 to provide the functionality ascribed to programmer 19 throughout this disclosure. For example, memory 72 may include a plurality of programs, where each program includes a parameter set that defines stimulation therapy.

User interface 74 may include a button or keypad, lights, a speaker for voice commands, a display, such as a liquid crystal (LCD), light-emitting diode (LED), or organic light-emitting diode (OLED). In some examples the display may be a touch screen. User interface 74 may be configured to display any information related to the delivery of stimulation therapy, identified patient behaviors, sensed patient parameter values, patient behavior criteria, or any other such information. User interface 74 may also receive user input via user interface 74. The input may be, for example, in the form of pressing a button on a keypad or selecting an icon from a touch screen.

Communication circuitry 76 may support wireless communication between an IMD and programmer 19, or between programmer 19 and charging device 20, under the control of processing circuitry 70. Communication circuitry 76 may also be configured to communicate with another computing device via wireless communication techniques, or direct communication through a wired connection. In some examples, communication circuitry 76 provides wireless communication via an RF or proximal inductive medium. In some examples, communication circuitry 76 includes an antenna, which may take on a variety of forms, such as an internal or external antenna.

Examples of local wireless communication techniques that may be employed to facilitate communication between programmer 19 and IMD 106 include RF communication according to the 802.11 or Bluetooth specification sets or other standard or proprietary telemetry protocols. In this manner, other external devices may be capable of communicating with programmer 19 without needing to establish a secure wireless connection.

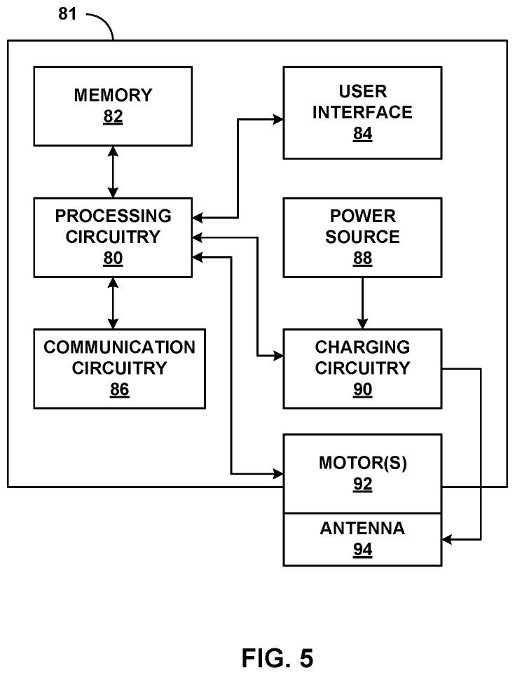

FIG. 5 is a block diagram of an example directional charging device 81. Charging device 81 may be substantially similar to charging device 20. In this manner, processing circuitry 80 may be similar to processing circuitry 50, memory 82 may be similar to memory 52, user interface 84 may be similar to user interface 54, communication circuitry 86 may be similar to communication circuitry 56, power source 88 may be similar to power source 60, and charging circuitry 90 may be similar to charging circuitry 58. However, antenna 94 may be a directional antenna configured to radiate RF energy in a specific direction. Therefore, one or more motors 92 may be coupled to antenna 94 and configured to move antenna 94 to a desired direction at which the device to be charged is located.

Processing circuitry 80 may control the one or more motors 92 to move antenna 94 to the desired direction for charging another device. Each of the one or more motors 92 may move the antenna about a respective axis. In this manner, multiple motors 92 can together move the antenna with multiple degrees of freedom in order to direct the RF energy from antenna 94 to the appropriate location. In this manner, charging device 81 may be configured to reduce unnecessary power for charging and limit the volume of tissue exposed to RF energy when charging an IMD.

In some examples, processing circuitry 80 may control the one or more motors 92 to scan or “hunt” for the best position at which to transmit RF energy to the controlled device. In this manner, processing circuitry 80 may control the one or more motors 92 to sweep through different positions and emit RF energy from antenna 94 at respective positions. Processing circuitry 80 may also receive charging information from the device to be charged, e.g., IMD 21A, that indicates the received power from the RF energy received. Since the received power would be a maximum when antenna 94 is directed to IMD 21A, processing circuitry 80 may identify the position at which the power was the greatest during the sweep and select that position for continued charging. Processing circuitry 80 may also continue to detect when the charging information from IMD 21A indicates the power decreases, and adjust one of motors 92 to move antenna 94 to a new position at which the IMD 21A has moved to. In this manner, charging device 81 may be able to find and track the position of IMD 21A implanted within a patient. For example, charging device 81 may be set on top of a table near a bed at which the patient is sleeping. Charging device 81 can then locate IMD 21A and adjust antenna 94 during patient sleep to maintain efficient charging and reduce tissue exposure to RF energy.

In other examples, a phased array antenna may be employed as antenna 94 with or without motors 92. For example, charging device 81 may modify the phase and/or amplitude of one or more antennas within the phased array to direct the RF signals to a target location. In this manner, motors or actuators may not be necessary. Instead of motors, processing circuitry 80 may control charging circuitry 90 to energies only those one or more antennas of the array to direct the RF signals to the target location or sweep through a plurality of locations using different subsets of the antennas in the array.

In some examples, charging device 81, or any other charging device described herein, may include one or more waveguides configured to direct RF signals towards a specific location, such as a medical device to be charged. The waveguide may be referred to as an electromagnetic feed line that is configured to conduct the RF signals towards the direction of interest. The waveguide may be constructed with walls that enable the RF signals to be reflected towards a specific direction. The waveguide may have a certain cross-sectional area and/or cross sectional dimensions relative to the frequency of the RF signal transmitted by antenna 94. For example, the waveguide may have an inner dimension of sufficient size to allow the wavelength of the RF signals to propagate within the waveguide (e.g., at least as large as one full wavelength of the RF signal). Antenna 94 may be disposed within, or adjacent, the waveguide. In this manner, charging device 81 can direct the RF energy from antenna 94 to the specific location of the IMD.

In some examples, the waveguide may enable charging device 81 to transmit RF signals with frequencies that would otherwise interfere with the operation of other devices in the environment around the patient. For example, charging device 81 may be configured to transmit RF signals in the frequency ranges of various consumer electronics devices if the RF signals are directed away from such devices. In addition, the waveguide may enable charging device 81 to reduce the power of transmitted RF signals because all, or substantially all, of the energy emitted by antenna 94 would be directed towards the IMD for charging. The use of the waveguide may reduce the power consumption of charging device 81 and reduce the RF energy transmitted to the patient during charging sessions. Alternatively, or additionally, the IMD may be capable of using larger amplitude RF signals by directing the RF signals through a waveguide because the directed RF signals will not otherwise interfere with the function of other devices near the patient. In other examples, charging device 81 may be configured to simultaneously charge different IMDs with different RF signal frequencies by using respective antennas and/or waveguides in order to direct the appropriate RF energy to the respective IMD.

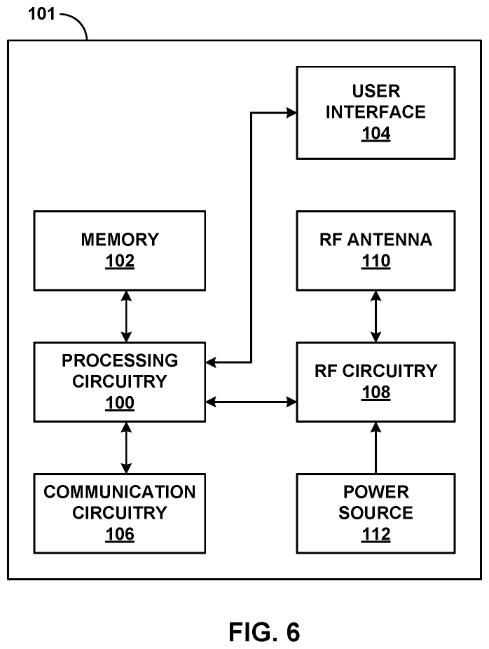

FIG. 6 is a block diagram of an example computer device 101 configured to emit RF signals for charging another device such as IMD 21A. Computing device 101 may take the form of any computing device that can transmit RF signals but may not be configured as a stand-alone charging device. In one example, computing device 101 may be a cellular phone (e.g., a smart phone) or Wi-Fi computing device (e.g., a tablet or notebook computer). In other examples, computing device 101 may be configured as a wearable device similar to a wrist watch, ankle charger, belt charger, or any other device that includes a strap or can be strapped adjacent to a medical device that should be charged. Computing device 101 may use one or more antennas, such as RF antenna 110, to radiate or transmit RF signals for charging another device such as IMD 21A.

Computing device 101 may be substantially similar to charging device 20 for purposes of charging. In this manner, processing circuitry 100 may be similar to processing circuitry 50, memory 102 may be similar to memory 52, user interface 104 may be similar to user interface 54, communication circuitry 106 may be similar to communication circuitry 56, power source 112 may be similar to power source 60, and RF circuitry 108 may be similar to charging circuitry 58. Antenna 110 may be similar to antenna 48, but antenna 110 may not be explicitly tuned to frequencies of the antenna of the device to be charged. Instead, processing circuitry 100 may control RF circuitry 108 to transmit RF energy from antenna 110. The device to be charged, such as IMD 21A, can then harvest the RF energy received from computing device 101.

In some examples, computing device 101 may broadcast RF energy in response to receiving a trigger signal. Computing device 101 may generate the trigger signal in response to determining a particular time of day (e.g., nighttime indicative of patient 12 sleeping), computer device 101 detects the presence of patient 12 in a room or otherwise within an envelope of computing device 101, power source 112 has a charge exceeding a predetermined threshold, RF antenna 110 is not currently used for other functions of computing device 101 (e.g., no other transmission and/or receiving of RF signals for communication), or computing device 101 has available computing and/or antenna bandwidth to transmit RF signals.

In some examples, computing device 101 may receive communications directly from IMD 14A or a user related to the function of IMD 14A or other IMD. For example, computing device 101 can receive a user input or signal from IMD 14A that has a sub-threshold battery voltage and requires recharging. Computing device 101 may thus begin broadcasting RF energy in an attempt to charge IMD 14A.

In some examples, computing device 101 may have reduced functionality such that computing device 101 is only configured to deliver RF energy for the purpose of recharging a medical device such as IMD 14A. In this case, computing device 101 may take the form of flat or curved rectangular device, or a cylindrical housing similar to a hockey puck. Computing device 101 that has reduced functionality may not include communication circuitry or extensive user interface options. For example, computing device 101 may only have a single power “on” and “off” toggle switch that initiates or terminates transmission of RF energy. Computing device 101 may thus be sized to include RF antenna 110, the power source (which may be rechargeable or non-rechargeable), and simple circuitry that supports turning RF energy transmission on and off. In this manner, computing device 101 may operate as a wireless recharging device that the patient may carry with them to recharge IMD 14A or other devices as needed.

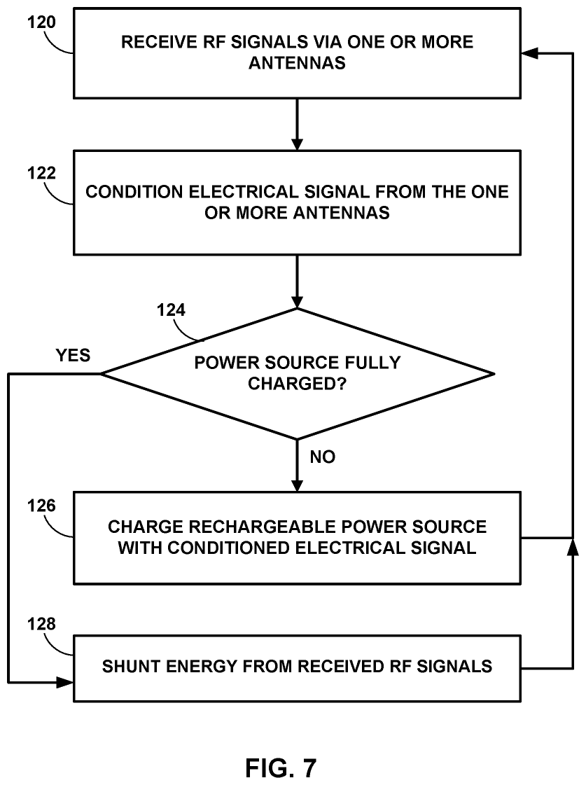

FIG. 7 is a flow diagram that illustrates an example technique for charging a power source of a medical device via received RF energy. Charging circuitry 26 and/or processing circuitry 22 of IMD 21A is described as generally performing the technique of example FIG. 7. However, in other examples, the technique of FIG. 7 may be performed by processing circuitry or device such as any IMD or device described herein. In addition, some functions of the process of FIG. 7 may be performed by distributed computing processes over at least two different devices.

As shown in the example of FIG. 7, charging circuitry 26 receives RF signals via one or more antennas such as antenna 28 (120). Charging circuitry 26 then conditions electrical signals from antenna 28 or other antennas (122). For example, charging circuitry 26 may convert the RF signals to a direct current for charging power source 24. If power source 24 is not fully charged, such as charged to a predetermined voltage threshold (“NO” branch of block 124), charging circuitry 26 charges rechargeable power source 24 with the conditioned electrical signal (126) and continues to receive RF signals (120). If the power source 24 is fully charged (“YES” branch of block 124), charging circuitry 26 may shunt received energy from the RF signals to the housing of IMD 21A (128) and continue to receive RF signals (120). In some examples, processing circuitry 22 may control charging circuitry 26 to perform any one or more of these functions.

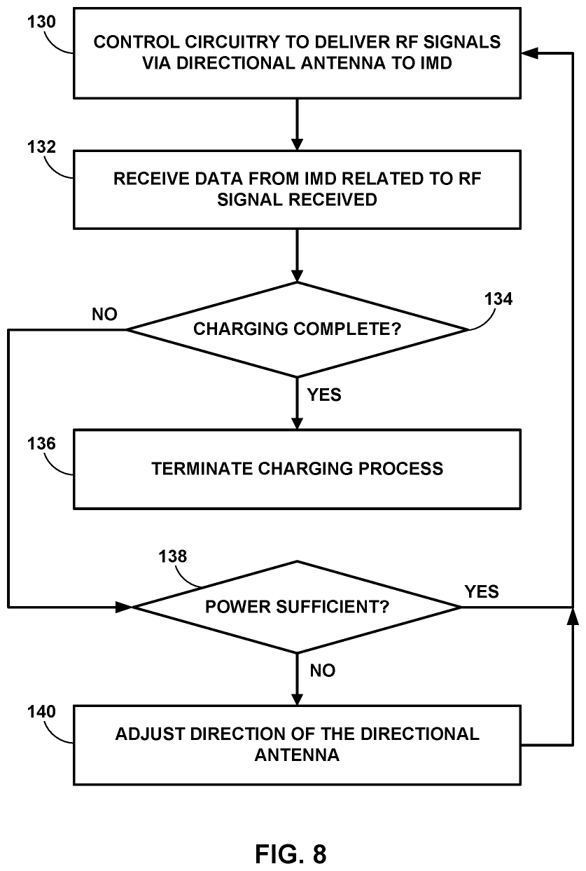

FIG. 8 is a flow diagram that illustrates an example technique for adjusting a position for an antenna that transmits RF energy for recharging a medical device. Charging circuitry 90 and/or processing circuitry 80 of charging device 81 is described as generally performing the technique of example FIG. 8. However, in other examples, the technique of FIG. 8 may be performed by processing circuitry or device such as any charging device or device described herein. In addition, some functions of the process of FIG. 8 may be performed by distributed computing processes over at least two different devices.

As shown in the example of FIG. 8, processing circuitry 80 controls charging circuitry 90 to deliver RF signals via directional antenna 94 (130). Directional antenna 94 may be configured to transmit RF signals in a particular direction as opposed to transmitting RF signals in all directions. Communication circuitry 86 receives data from an IMD, such as IMD 21A, related to the RF signal energy received by IMD 21A (132). This data may be charging information or any other information associated with the received RF energy from charging device 81. If the charging information from IMD 21A indicates that charging is the rechargeable power source is complete (“YES” branch of block 134), processing circuitry 80 terminates the charging process and transmission of RF energy (136).

If the charging information from IMD 21A indicates that charging is the rechargeable power source is not complete (“NO” branch of block 134), processing circuitry 80 determines if the power received by IMD 21A is sufficient (138). If the power is sufficient (“YES” branch of block 138), charging circuitry 90 continues to deliver RF signals (130). If the power is not sufficient (“NO” branch of block 138), processing circuitry 80 controls one or more of motors 92 to adjust the direction of the directional antenna (140). Processing circuitry 80 may determine whether or not the power received by IMD 21A is sufficient by tracking a trend of power received. For example, if processing circuitry 80 receives charging information that indicates that the power has been reduced, processing circuitry may determine that the directional antenna 94 is not positioned correctly to direct RF energy to IMD 21A. Therefore, processing circuitry 80 may begin to move antenna 94 in an attempt to more closely align antenna 94 to IMD 21A. This may be an iterative process in which processing circuitry 80 moves antenna 94 and evaluates how the move affected the RF energy received by IMD 21A. In other examples, the process of FIG. 8 may include processing circuitry 80 sweeping antenna 94 through a plurality of positions and analyzing received charging information from IMD 21A to determine which position processing circuitry 80 should use for transmitting RF energy to IMD 21A.

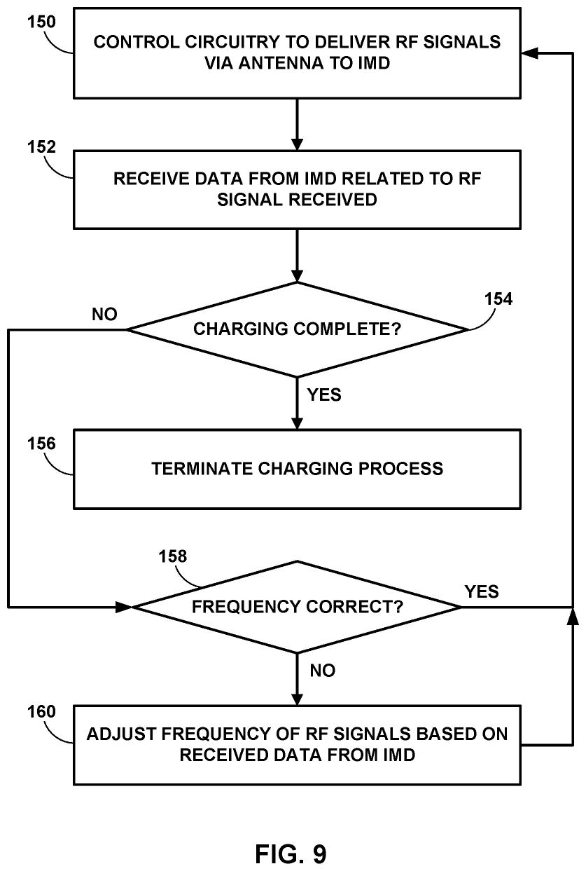

FIG. 9 is a flow diagram that illustrates an example technique for transmitting RF energy at different frequencies for recharging a medical device. Charging circuitry 58 and/or processing circuitry 50 of charging device 20 is described as generally performing the technique of example FIG. 9. However, in other examples, the technique of FIG. 9 may be performed by processing circuitry or device such as any charging device or device described herein. In addition, some functions of the process of FIG. 9 may be performed by distributed computing processes over at least two different devices.

As shown in the example of FIG. 9, processing circuitry 50 controls charging circuitry 58 to deliver RF signals via antenna 48 (150). Communication circuitry 56 receives data from an IMD, such as IMD 21A, related to the RF signal energy received by IMD 21A (152). This data may be charging information or any other information associated with the received RF energy from charging device 20. If the charging information from IMD 21A indicates that charging is the rechargeable power source is complete (“YES” branch of block 154), processing circuitry 50 terminates the charging process and transmission of RF energy (156).

If the charging information from IMD 21A indicates that charging is the rechargeable power source is not complete (“NO” branch of block 154), processing circuitry 50 determines if the frequency is appropriate based on the power received by IMD 21A is sufficient (158). RF signals may shift in frequency (e.g., reduce the frequency) as the signals travel through tissue or pass through other objects. If the frequency of the RF signals is not appropriate to the configuration of the antenna from IMD 21A, IMD 21A will receive less power. Therefore, processing circuitry 50 may determine to increase the frequency of RF signals in order for the antenna of IMD 21A, for example, to receive signals having a frequency to which the antenna is configured to receive which should be realized as greater power. If the frequency of RF signals is correct (“YES” branch of block 158), charging circuitry 58 continues to deliver RF signals (150). If the frequency is not correct (“NO” branch of block 158), processing circuitry 50 adjusts the frequency of the RF signals based on the data received from IMD 21A (150). For example, processing circuitry 50 may increase the frequency of RF signals and/or decrease the frequency of RF signals in an iterative manner to track which frequencies result in the greatest power of RF energy received by IMD 21A.

As described herein, charging device 20 can perform frequency hopping to change the frequency of RF energy transmitted for the purposes of charging IMD 21A. In some examples, charging device 20 may estimate whether to increase or decrease the frequency shift for subsequent RF signals based on data from IMD 21A or charging levels. Charging device 20 may periodically adjust the RF signal frequency in order to achieve improved charging at IMD 21A. Charging device 20 may monitor the relative charging rates at IMD 21A based on received charging information from IMD 21A. Increases in charging rates in response to a change in RF signal frequency may indicate that the previous change in frequency was an improvement. Charging device 20 may thus further change the RF signal frequency in that same direction (e.g., continue to increase the frequency or continue to decrease the frequency) until the charging rate is reduced at IMD 21A. Conversely, decreases in charging rates in response to a change in RF signal frequency may indicate that the previous change in frequency was shifting the RF signal frequency in the wrong direction. Charging device 20 can responsively adjust the frequency of the RF signals in the opposite direction than what was performed previously. By using these frequency shifting techniques, charging device 20 may increasing the efficiency of energy transfer to IMD 21A in order to reduce the recharge time for IMD 21A and reduce energy consumption of charging device 20.

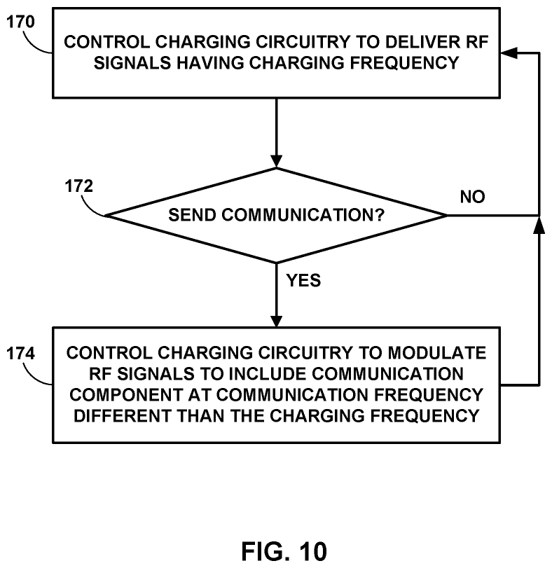

FIG. 10 is a flow diagram that illustrates an example technique for transmitting RF energy and communication information at different frequencies. Charging circuitry 58 and/or processing circuitry 50 of charging device 20 is described as generally performing the technique of example FIG. 10. However, in other examples, the technique of FIG. 10 may be performed by processing circuitry or device such as any charging device or device described herein. In addition, some functions of the process of FIG. 10 may be performed by distributed computing processes over at least two different devices.

As shown in the example of FIG. 10, processing circuitry 50 controls charging circuitry 58 to deliver RF signals via antenna 48 having a charging frequency (170). If processing circuitry 50 determines that no communication needs to be sent, (“NO” branch of block 172), processing circuitry 50 continues to control charging circuitry 58 to deliver charging frequency RF signals via antenna 48 (170). If processing circuitry 50 determines that communication should be sent (“YES” branch of block 172), processing circuitry 50 controls charging circuitry 58 to modulate RF signals to include a communication component at a communication frequency that is different from the charging frequency (174). This change may be due to the device, such as IMD 21A, receiving communication over different frequencies than the charging energy. In some examples, processing circuitry 50 may control charging circuitry 58 to adjust a characteristic of a tuning circuit or other element associated with antenna 48 to achieve the different communication frequency. In other examples, processing circuitry 50 may control the communication information to be transmitted via a different antenna configured to transmit the communication information in the communication frequency.

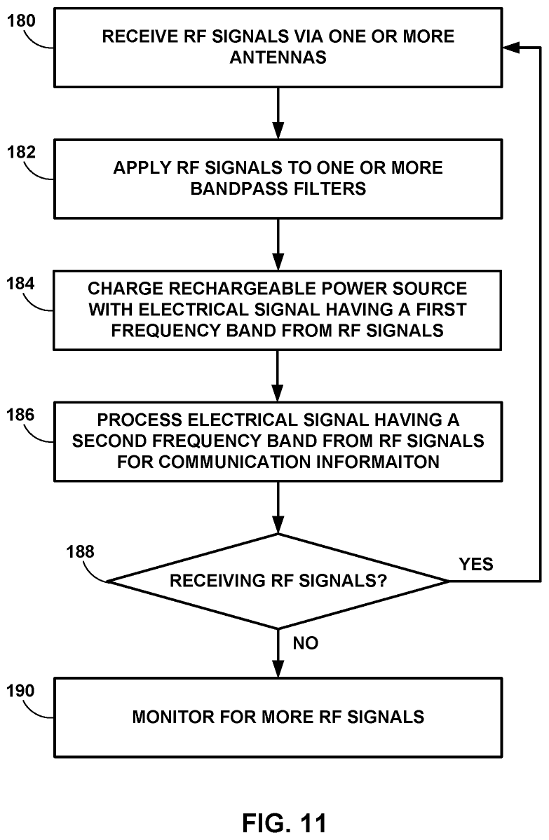

FIG. 11 is a flow diagram that illustrates an example technique for separating charging power from communication information using one or more bandpass filters. FIG. 11 may be associated with the receiving device from charging device 20 transmitting charging power and communication information in FIG. 10. Charging circuitry 26, processing circuitry 22, and/or signal circuitry 37 of IMD 21B is described as generally performing the technique of example FIG. 11. However, in other examples, the technique of FIG. 11 may be performed by processing circuitry or device such as any IMD or device described herein. In addition, some functions of the process of FIG. 11 may be performed by distributed computing processes over at least two different devices.

As shown in the example of FIG. 11, signal circuitry 37 receives RF signals via one or more antennas such as antenna 28 (180). Signal circuitry 37 then applies the RF signals to one or more bandpass filters (182). In some examples, IMD 21B may include one bandpass filter to recover the communication information from the RF signals. In other examples, IMD 21B may include two bandpass filters to recover respective communication information and charging energy. Charging circuitry 26 then recharges power source 24 with an electrical signal having the first frequency, or band of frequencies, from the RF signals (184). Communication circuitry 39 also processes the electrical signal having a second frequency, or frequency band, from the RF signals that includes the communication information (186). In other examples, the steps 184 and 186 may be switched. For example, signal circuitry 37 may remove the communication information from the RF signals and then transmit any remaining frequencies and energy to charging circuitry 26. In other examples, signal circuitry may include a directional coupler or other device to siphon off a small portion of the RF energy and feed that smaller portion of RF energy through a bandpass filter to isolate the communication signal for communication circuitry 39. These processes, alone or in some combination, may retain more RF energy for charging. Communication circuitry 39 may then send the communication information to processing circuitry 22. If signal circuitry 37 determines that RF signals are still being received (“YES” branch of block 188), signal circuitry 37 continues to receive RF signals (180). If signal circuitry 37 determines that RF signals are no longer being received (“NO” branch of block 188), signal circuitry 37 monitors for additional RF signals (190).

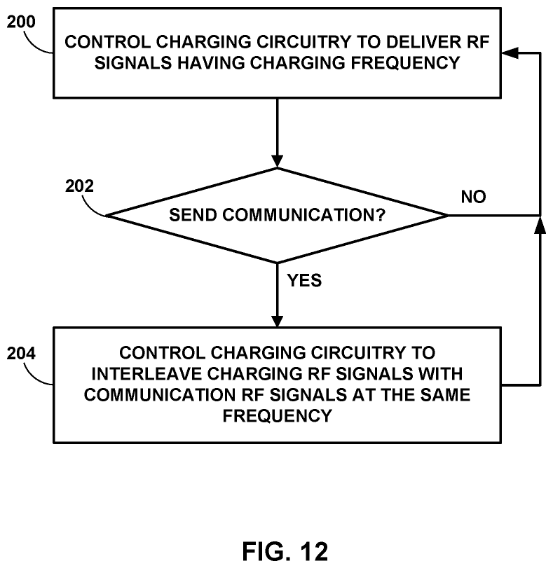

FIG. 12 is a flow diagram that illustrates an example technique for transmitting interleaved RF energy and communication information at the same frequency. Charging circuitry 58 and/or processing circuitry 50 of charging device 20 is described as generally performing the technique of example FIG. 12. However, in other examples, the technique of FIG. 12 may be performed by processing circuitry or device such as any charging device or device described herein. In addition, some functions of the process of FIG. 12 may be performed by distributed computing processes over at least two different devices.

As shown in the example of FIG. 12, processing circuitry 50 controls charging circuitry 58 to deliver RF signals via antenna 48 having a charging frequency (200). If processing circuitry 50 determines that no communication needs to be sent, (“NO” branch of block 202), processing circuitry 50 continues to control charging circuitry 58 to deliver charging frequency RF signals via antenna 48 (200). If processing circuitry 50 determines that communication should be sent (“YES” branch of block 202), processing circuitry 50 controls charging circuitry 58 to interleave charging RF signals with communication RF signals at the same frequency (204). Processing circuitry 50 may control the interleaving process according to a predetermined schedule so that IMD 21A, for example, can decode the RF signals appropriately over time. In other examples, processing circuitry 50 may control charging circuitry to add a flag or indicator in the RF signals that indicates when upcoming RF signals are intended for charging or include communication information.

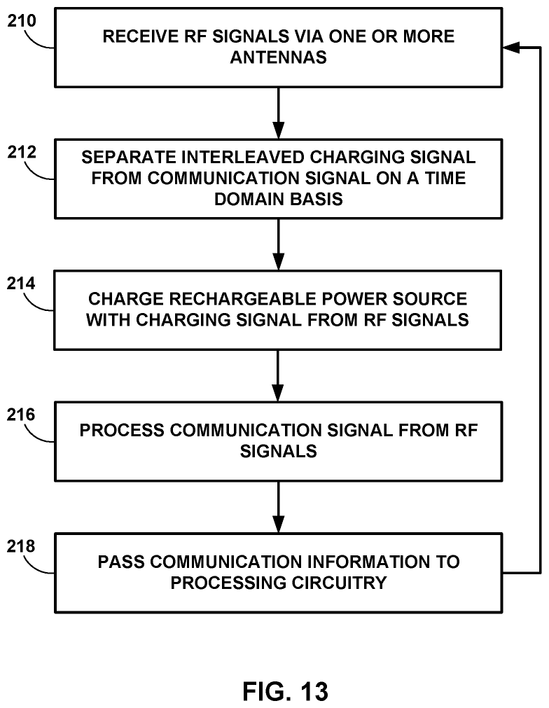

FIG. 13 is a flow diagram that illustrates an example technique for separating interleaved charging power and communication information from received RF energy. FIG. 13 may be associated with the receiving device from charging device 20 transmitting charging power and communication information in FIG. 12. Charging circuitry 26, processing circuitry 22, and/or signal circuitry 37 of IMD 21B is described as generally performing the technique of example FIG. 13. However, in other examples, the technique of FIG. 13 may be performed by processing circuitry or device such as any IMD or device described herein. In addition, some functions of the process of FIG. 13 may be performed by distributed computing processes over at least two different devices.

As shown in the example of FIG. 13, signal circuitry 37 receives RF signals via one or more antennas such as antenna 28 (210). Signal circuitry 37 then separates the interleaved charging signal from the communication signal on a time domain basis (212). For example, signal circuitry 37 may follow a predetermined schedule of interleaving when communication information is included in the received RF signals. In other examples, signal circuitry 37 may follow indicators embedded in the RF signals indicating when the RF signals include communication information. Signal circuitry 37 then passes the charging signals to charging circuitry 26 for recharging power source 24 (214). Signal circuitry 37 then also passes communication signals to communication circuitry 39 for processing (216). Communication circuitry 39 then passes the communication information to processing circuitry 22 (218).

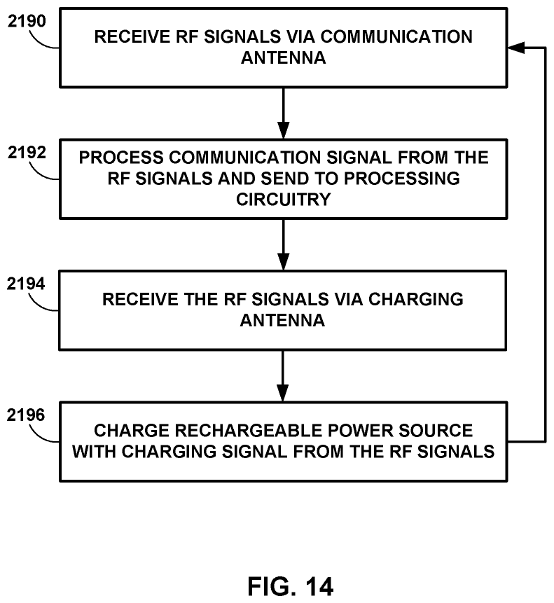

FIG. 14 is a flow diagram that illustrates an example technique for obtaining charging power and communication from transmitted RF energy. Charging circuitry 26, processing circuitry 22, and/or communication circuitry 36 of IMD 21A is described as generally performing the technique of example FIG. 14. However, in other examples, the technique of FIG. 14 may be performed by processing circuitry or device such as any IMD or device described herein. In addition, some functions of the process of FIG. 14 may be performed by distributed computing processes over at least two different devices.

As shown in the example of FIG. 14, IMD 21A can receive communication data and charging power from the same RF signals (e.g., the same frequency or frequencies of the RF energy transmitted by an external device). Communication circuitry 36 receives RF signals via one or more communication specific antennas (2190). Communication circuitry 36 can then process the communication signal from the RF signals and send to processing circuitry 22 as appropriate (2192).

IMD 21A also, either simultaneously or on an interleaved basis, receives RF signals via one or more charging specific antennas (e.g., antenna 28) (2194). Again, the received RF signals are the same signals received by the antenna of communication circuitry 36. Charging circuitry 26 then charges power source 24 with the RF energy harvested from the received RF signals (2196). This process can then be repeated as IMD 21A is operational. Again, IMD 21A can interleave the communication and charging receiving of RF signals or operate charging circuitry 26 and communication circuitry 36 to simultaneously and/or independently, receive the RF energy at the same frequencies.

In this manner, a charging device, such as charging device 20, can transmit a single RF signal for both communication with IMD 21A and charging of IMD 21A. In one example, charging device 20 may merely increase the amplitude of the RF signals being transmitted to increase the available energy to harvest for charging purposes. However, charging device 20 may still ensure that the total power transmitted to IMD 21A remains within safe tolerances to reduce the likelihood of any adverse reaction to the delivered RF energy. In other examples, communication circuitry 36 may directly harvest energy from the RF signals. For example, communication circuitry 36 may direct a portion of the current generated from the communication antenna to charging power source 24 while retaining a remaining portion of the current for detecting communication information.

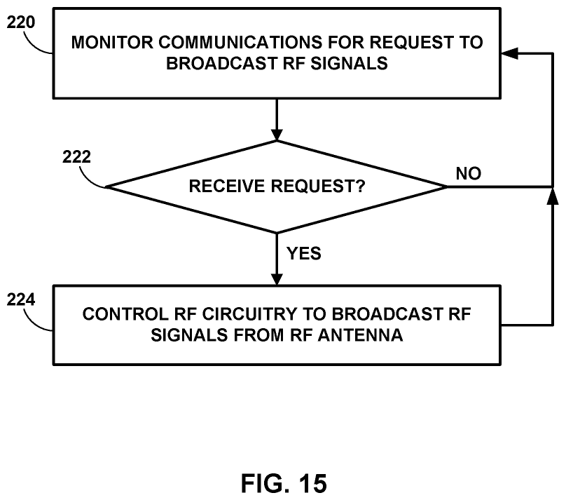

FIG. 15 is a flow diagram that illustrates an example technique for broadcasting RF energy in response to receiving a request to charge. RF circuitry 108 and/or processing circuitry 100 of computing device 101 is described as generally performing the technique of example FIG. 15. However, in other examples, the technique of FIG. 15 may be performed by processing circuitry or device such as any charging device (e.g., charging device 20 or 81) or device described herein. In addition, some functions of the process of FIG. 15 may be performed by distributed computing processes over at least two different devices.

As shown in the example of FIG. 15, processing circuitry 100 monitors communications for a request to broadcast RF signals (220). In other examples, the request may be a trigger signal generated in response to an internally detected event, condition, or timer. For example, processing circuitry 100 may determine that antenna 110 is not being utilized by any other processes, power source 112 contains sufficient voltage to transmit RF energy, or any other indicator that transmission of RF energy is appropriate is received. If processing circuitry 100 determines that no request has been received (“NO” branch of block 222), processing circuitry 100 continues to monitor for any requests (220). If processing circuitry 100 determines that communication should be sent (“YES” branch of block 222), processing circuitry 100 controls RF circuitry 108 to broadcast RF signals from RF antenna 110 for charging another device, such as IMD 21A (224).

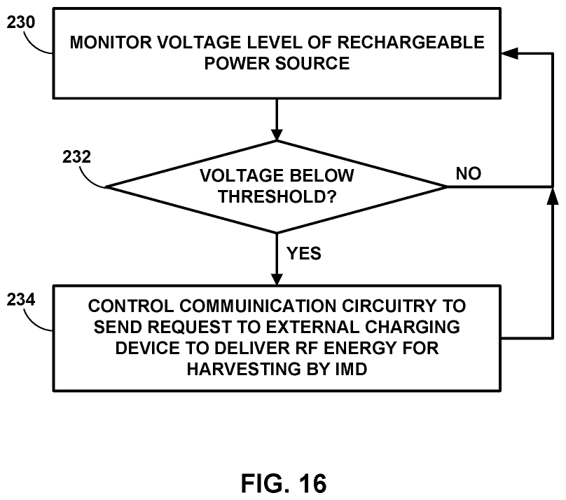

According to the techniques of FIG. 15, computing device 101 or any charging device can listen for requests from an IMD for recharging power and start broadcasting RF energy for charging the IMD in response to receiving the request to start. FIG. 16 below illustrates an example technique for the IMD to determine when to transmit a request for RF energy broadcasting from one or more charging devices. In some examples, computing device 101 may only respond to the request to transmit RF energy when computing device 101 is close enough to the IMD that the request can be “heard” or received. In this manner, only those charging devices close enough to the IMD will broadcast RF energy as the patient moves around an environment (e.g., their home or other location). Computing device 101 may continue to broadcast RF energy as long as the request is continually or periodically received from the IMD. In this manner, if the communication from the IMD is broken or computing device 101 simply does not receive additional requests after a predetermined period of time, computing device 101 can responsively shut down or terminate broadcasting RF energy. For example, computing device 101 may no longer request the request when IMD has determined that the rechargeable power supply has reached a full charge. In this manner, the IMD and one or more charging devices, such as computing device 101 and/or charging device 20, can operate autonomously without any interaction from the patient to recharge the IMD when needed while reducing the amount of time charging devices are broadcasting RF energy around the patient.

FIG. 16 is a flow diagram that illustrates an example technique for transmitting a request to transmit RF energy in response to a trigger event. FIG. 16 may be associated with the receiving device from computing device 101 transmitting charging power in FIG. 15. Charging circuitry 26 and/or processing circuitry 22 of IMD 21A is described as generally performing the technique of example FIG. 16. However, in other examples, the technique of FIG. 16 may be performed by processing circuitry or device such as any IMD or device described herein. In addition, some functions of the process of FIG. 16 may be performed by distributed computing processes over at least two different devices.

As shown in the example of FIG. 16, processing circuitry 22 monitors the voltage level of rechargeable power source 24 (230) within the IMD. The voltage level may be indicative of the remaining power available from power source 24. For example, below a voltage threshold level, power source 24 may only be able to provide operational power to IMD 21A for a short time. If processing circuitry 22 determines that the voltage of power source 24 is not below the threshold (“NO” branch of block 232), processing circuitry 22 may continue to monitor the voltage level (230). If processing circuitry 22 determines that the voltage of power source 24 is below the threshold (“YES” branch of block 232), processing circuitry 22 may control communication circuitry 36 to send a request to an external charging device (e.g., charging device 20 or computing device 101) to deliver RF energy for harvesting by IMD 21A (234). In this manner, if IMD 21A is not able to harvest sufficient amounts of RF energy from ambient RF signals in the environment around IMD 21A, IMD 21A can request a charging device to provide additional RF energy. IMD 21A may continue to transmit the request (e.g., continually or at some predetermined periodic rate) to one or more charging devices, such as any charging devices close enough to receive the request, as long as RF energy is still required to recharge power source 24. In this manner, RF energy will no longer be broadcast if IMD 21A stops requesting the RF energy. Alternatively, IMD 21A may transmit a termination request when recharge is complete.

In certain situations, transmission of RF energy to IMD 21A may interfere with one or more functions of IMD 21A. For example, IMD 21A may be configured to sense physiological signals from the patient, such as electrical signals using electrodes of an implanted lead or other sensor. In the case where IMD 21A senses electrical brain activity (e.g., local field potentials (LFPs), electroencephalograms (EEGs), etc.) to detect seizures or other brain function, RF energy may interfere with the detection of these physiological electrical signals. The RF energy may create artifacts that interfere with the identification of physiological events in the sensed data or the RF energy may introduce noise that has a larger amplitude of the sensed signals. These sensing issues may occur or be amplified for lower RF signal frequencies and higher sensing frequencies. To avoid interference with sensing function, IMD 21A and/or charging device 20 may time the transmission of RF energy to avoid sensing activity. IMD 21A may time the request to charging device 20 for RF energy based on sensed signals or known period of sensing, of charging device 20 may transmit RF energy in accordance with a schedule at which IMD 21A performs sensing functions. For example, if IMD 21A detects LFPs indicative of a non-symptomatic state or that sensing is not required, IMD 21A may transmit a request to charging device 20 to transmit RF energy. In some examples, IMD 21A may specific a duration for transmission of RF energy. In one example, IMD 21A may employ such a procedure before step 234 in FIG. 16. IMD 21A may withhold the request to transmit the request for RF energy until after a particular sensing or stimulation window has terminated. In this manner, RF energy transmission may periodically turn off, or reduce in power, in order to avoid interference with IMD 21A sensing or other functions.

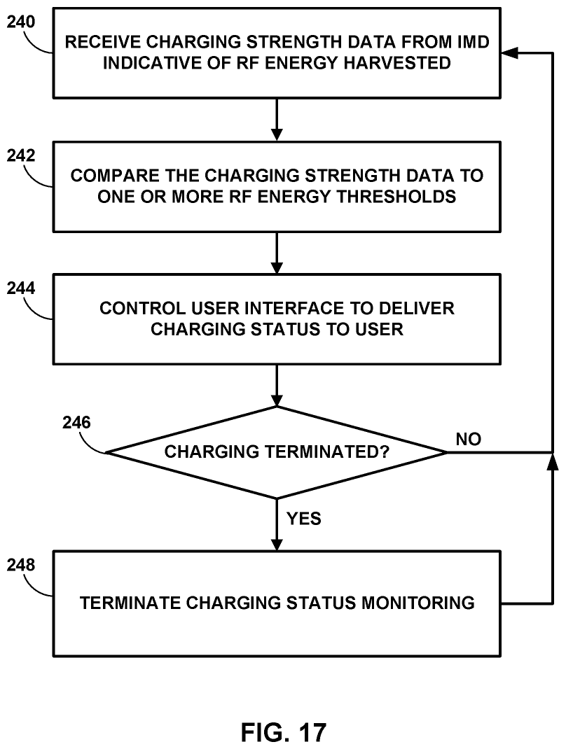

FIG. 17 is a flow diagram that illustrates an example technique for providing feedback to a user regarding RF energy reception status. Processing circuitry 50 of charging device 20 is described as generally performing the technique of example FIG. 17. However, in other examples, the technique of FIG. 17 may be performed by processing circuitry or device such as any charging device or device described herein. In addition, some functions of the process of FIG. 17 may be performed by distributed computing processes over at least two different devices.

As shown in the example of FIG. 17, processing circuitry 50 receives charging strength data (e.g., which may be part of charging information) from IMD 21A that is indicative of the RF energy harvested by IMD 21A (240). The charging strength data may be indicative of a current delivered to power source 24 and/or the charging rate of power supply 24. Processing circuitry 50 may then compare the charging strength data to one or more RF energy thresholds (242). The one or more RF energy thresholds may correspond to respective charging levels or charging efficiencies. For example, if there are two charging levels (e.g., bad and good), a single RF energy threshold may correspond to the charging level that separates the bad from the good charging. Three charging levels, e.g., low, medium, and high, may be separated by two thresholds between the low and medium and between the medium and high levels.

Each of these charging levels may be indicative of the signal strength or the RF energy actually harvested by IMD 21A. Since the signal strength may be affected by material (e.g., structures or patient tissues) or lack thereof between IMD 21A and charging device 20, the user may benefit from feedback indicating the signal strength of RF energy being harvested. For example, the user may move to a different position or move charging device 20 to a different position in order to improve the transmission efficiency to IMD 21A. Processing circuitry 50 may control user interface 54 to deliver the charging status to the user based on the comparison of the charging strength data to the one or more thresholds (or an equation or any other analysis) (244). For example, processing circuitry 50 may control user interface 54 to display text or numeral(s) representative of the relative signal strength, display a color representative of the relative signal strength, emit an audible indication of the signal strength, or provide any other feedback. In some examples, processing circuitry 50 may update the signal strength feedback in real time in order for the user to understand how changes to position or distance to charging device 20 can increase or reduce the power of the RF energy received by IMD 21A.

If processing circuitry 50 determines that charging has not been terminated (“NO” branch of block 246), processing circuitry 50 continues to receive charging strength data from IMD 21A (240). If processing circuitry 50 determines that charging has been terminated (“YES” branch of block 246), processing circuitry 50 terminates the charging status monitoring and display (248). Although charging device 20 is described as providing the user feedback regarding RF signal strength, any device may be configured to provide this feedback via one or more user interfaces. For example, a smart phone, smart watch, or other computing device may receive charging information from IMD 21A, charging device 20, or any other device and responsively provide the feedback via one or more notifications or user interface elements that may include optical, audible, and/or tactile modalities.

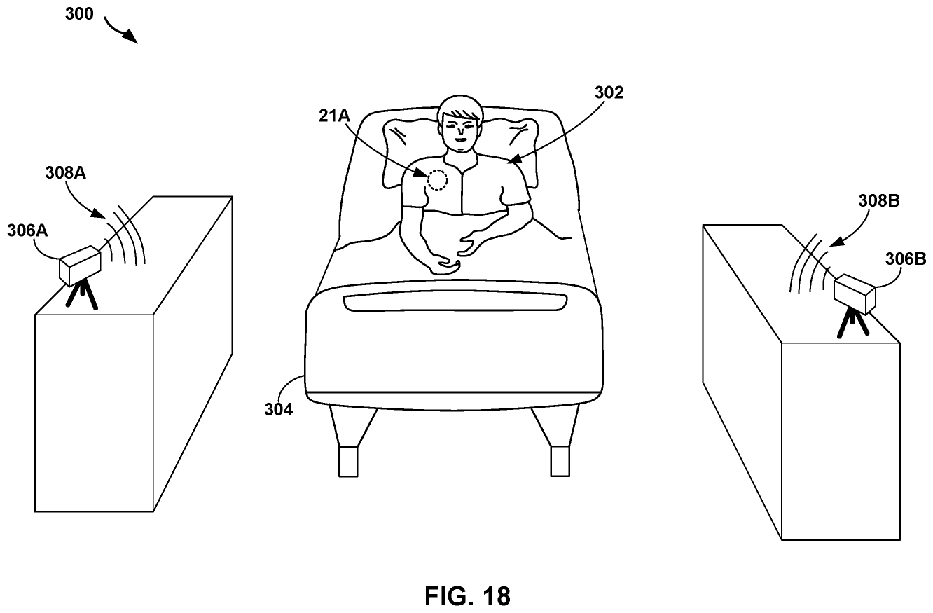

FIG. 18 is a conceptual diagram illustrating an example array of RF energy sources for transmitting RF energy that can be harvested by IMD 21. As shown in the example, of FIG. 18, multiple charging devices 306A and 306B (collectively “charging devices 306”) can be configured to deliver RF energy to an environment 300 within which patient 302 is disposed. Patient 302 is shown in bed 304, but patient 304 may sit in a different piece of furniture or even be ambulatory during RF harvesting. Patient 302 may have IMD 21A which can harvest RF energy from available RF signals. However, a single source of RF energy may not be able to provide sufficient energy for charging based on the location of IMD 21A with respect to the charging device. Multiple charging devices 306 (e.g., two or more charging devices) places around an environment 300, such as a room or house, may improve the likelihood that IMD 21A can harvest more RF energy to recharge power source 24. For example, the body of patient 302 or other device in environment 300 may shield IMD 21A from one or more charging devices 306. Charging devices 306 may be similar to any of charging device 20 or 81 described herein.

Each of charging devices 306A and 306 emit RF energy 308A and 308B, respectively. Although charging devices 306 look identical, different charging devices 306 may be different types of devices (e.g., dedicated charger or cellular phone) or even transmit different power and/or frequencies of RF energy. In any case, multiple charging devices 306 may increase the available RF power and/or time during which IMD 21A can receive power from at least one charging device 306. In addition, each charging device 306 may be limited to a magnitude of RF power to prevent unsafe levels of RF energy to patient 302. Multiple charging devices 306 may enable each charging device to remain under any such limit while still providing for a larger area of space blanketed with RF energy for harvesting.

In some examples, each of charging devices 306 may be synchronized in order to establish a phased array of RF transmission devices. The phased array may be synchronized in order to ensure that the RF signals from RF energy 308A and 308B are in phase (or partially in phase as appropriate). Otherwise, out of phase RF signals may create nulls at locations of IMD 21A and prevent the harvesting of any RF energy at those locations. Charging devices 306 may be initially synchronized or continually or periodically in communication with each other in order to maintain the phased array. In some examples, IMD 21A may transmit a timing signal received by charging devices 306 directly or via some other networked device in communication with IMD 21A and charging devices 306. In some examples, charging devices 306 may adjust the phase of emitted RF energy to coordinate with the location of IMD 21A as IMD 21A moves with respect to each charging devices. For example, IMD 21A may broadcast the received RF energy and charging devices 306 may responsively adjust the phase of emitted RF energy 308A and 308B in an attempt to increase the received RF energy at the location of IMD 21A.

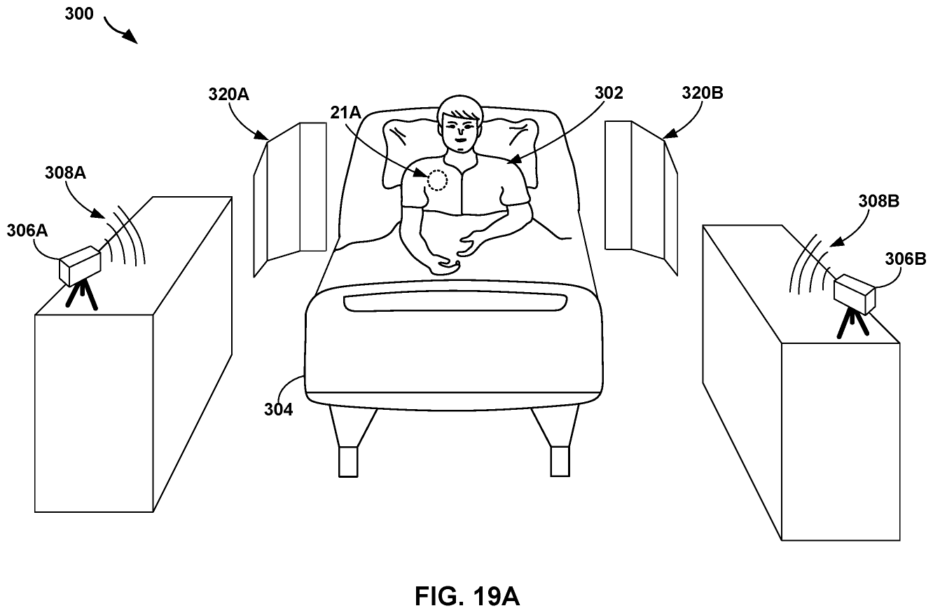

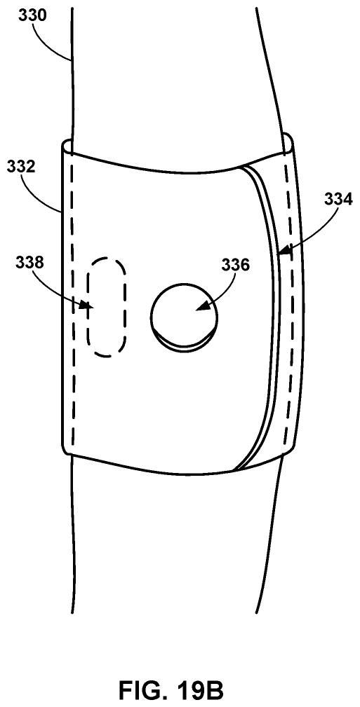

FIGS. 19A and 19B are a conceptual diagram illustrating example reflectors 320A and 320B configured to reflect RF energy to an implantable medical device such as IMD 21A. As shown in the example of FIG. 19A, patient 320 is positioned in bed 304 similar to FIG. 18. Charging devices 306 transmit RF energy 308A and 308B, but some of the RF energy may not reach IMD 21A. Therefore, reflectors 320A and 320B (collectively, “reflectors 320”) are positioned within environment 300 to reflect at least a portion of RF energy 308A and 308B back towards IMD 21A. Reflectors 320 may be constructed of a metal alloy, metal, or other material configured to reflect RF energy.

One or more reflectors, such as reflectors 320, may be positioned on an opposite side of patient 320 from charging devices 306 or at various positions around environment 300 to reflect at least a portion of RF energy 308A and 308B to improve the likelihood that the RF energy reaches IMD 21A. Reflectors 320 are shown positioned opposite of respective charging devices 306. For example, reflector 320B may reflect RF energy 308A back towards IMD 21A, and reflector 320A may reflect RF energy 308B back towards IMD 21A. Although two reflectors 320 are shown, only a single reflector or three or more reflectors may be disposed in environment 300 in other examples. Reflectors 320 may be freestanding on the floor, attached to a wall, or placed on a shelf. The positions of reflectors 320 may be selected according to the position of charging devices 306 and common locations for patient 302. In other examples, reflectors 320 may be disposed within or under a pillow, within or under a mattress of bed 304, or at any other location appropriate for reflecting RF energy back to IMD 21A.

Reflectors 320 may be utilized with directional charging devices, such as charging device 81, that provide a narrow beam of RF energy. In this manner, reflectors 320 may enable the narrow beam of RF energy to pass through more space and improve the likelihood that the RF energy is received by IMD 21A. In other examples, reflectors 320 may be used with non-directional charging devices to further improve RF energy coverage over the volume of environment 300.

As shown in the example of FIG. 19B, reflector 332 is wrapped around leg 330 of the patient. The patient may have an IMD 338 which may be similar to IMD 21A and positioned to provide stimulation to a tibial nerve within leg 330. Reflector 332 may be flexible or include a plurality of creases that support bending to enable reflector 332 to wrap fully or partially around leg 330. Outer edge 334 of reflector 332 overlaps a portion of reflector 332 to encompass leg 330.

Reflector 332 may define an orifice 336 that is an opening through reflector 332. The orifice may be sized to allow RF energy to enter through orifice 336 and reflect back and forth within reflector 332 until the antenna of IMD 338 can absorb the RF energy or the RF energy exits through orifice 336. In this manner, reflector 332 may improve the efficiency of absorption of the RF energy by IMD 338. Reflectors similar to reflector 332 may be provided for other parts of the body, such as the arm or torso, according to where the IMD is implanted. For example, the patient may wear a coat that includes a reflective coating to improve the coverage of RF energy and likelihood that RF energy reaches the antenna of the IMD. In some examples, wide band RF energy may be utilized with a reflector such as reflector 332 to accommodate the varying frequency shifts that will occur as RF signals travel through more tissue of leg 330.

Reflector 332 may provide some advantages. Energy transfer through tissue may be limited to certain amplitudes, frequencies, or other parameters, in order to prevent the energy transfer from exceeding a safe limit for the patient. In this manner, reflector 332 may enable lower amplitudes of RF energy to be delivered because more energy from the delivered RF energy at the lower amplitudes ends up being absorbed by the antenna of IMD 338. Therefore, using reflector 332 or other types of reflectors may enable the charging device to provide RF energy below the safety threshold and transfer a larger proportion of RF energy to IMD 338 for a more complete recharge of IMD 338 even at the lower amplitudes of RF energy.

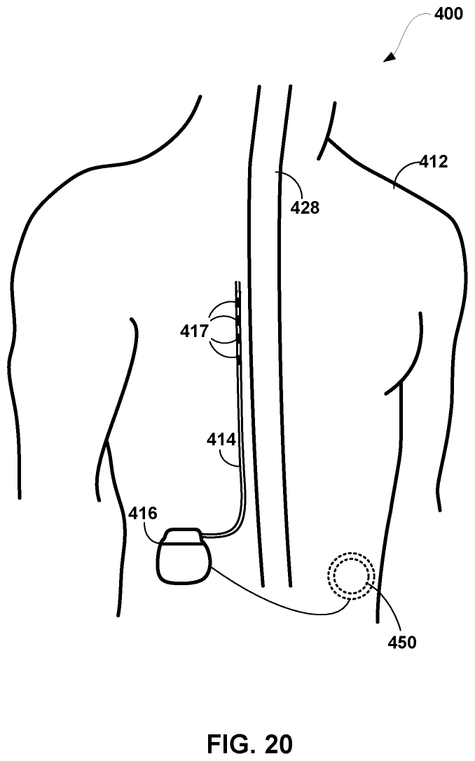

FIG. 20 is a conceptual diagram illustrating an example IMD 416 coupled to a separate antenna 450. IMD 416 may be similar to other IMDs described herein, such as IMD 21A, and includes a rechargeable power source. IMD 416 is coupled to lead 414 that carries electrodes. Lead 414 may tunnel through tissue of patient 412 from along spinal cord 428 to a subcutaneous tissue pocket or other internal location where IMD 416 is disposed. IMD 416 may deliver electrical stimulation to spinal cord 428 and/or other locations within patient 412. However, in cases where IMD 416 is implanted to provide DBS therapy or other therapies to other locations, including one or more nerves of the pelvic floor and the tibial nerve, IMD 416 may use antennas to harvest RF energy.

For example, system 400 may include one or more tethered antennae connected to IMD 416. IMD 416 may include one or more ports, e.g., one or more connectors on the header of IMD 416, to connect tethered antenna 450 to the circuitry of IMD 416. Tethered antenna 450 may allow the antenna to be connected to IMD 416 through a port or header of IMD 416. Tethered antenna 450 may be configured for harvesting RF signals for recharging or for receiving or transferring communication data. Tethered antenna 450 may be implanted proximal to IMD 416 in some examples, while in other examples, tethered antenna 450 may be implanted in a different location. In some examples, tethered antenna 450 may provide advantages compared to other antenna configurations, such as an internal antenna, or an antenna on the external surface of IMD 416.

In some examples, tethered antenna 450 may be placed close to the surface of the skin and therefore may be subject to less of an impact from tissue absorption of RF energy, and thus may be more effective than an antenna housed within the housing of IMD 416. Tethered antenna 450 may include one or more fixation elements (e.g., tines or barbs), facilitate sutures, or provide another anchor mechanism in order to anchor tethered antenna 450 in tissue may remain at the implanted location even if IMD 416 migrates or flips within the tissue pocket. In other examples, tethered antenna 450 may have a percutaneous connection to IMD 416 such that tethered antenna 450 is outside of patient 412 while IMD 416 remains within patient 412. In addition, when harvesting in different radio environments, different antennae, e.g., different shapes, sizes and so on may provide more options depending on the radio environment. For example, tethered antenna 450 may be larger than the housing of IMD 416 and therefore might be more effective in some environments. Alternatively, tethered antenna 450 may include multiple different antennas in order to increase the area by which IMD 450 can absorb RF energy. Tethered antenna 450 may be any shape, e.g., a fractal shape, in some examples.

The following examples are described herein. Example 1: An implantable medical device comprising: a rechargeable power supply; an antenna configured to receive radio frequency (RF) energy having one or more frequencies within at least one of a first range from 1 MHz to 20 MHz or a second range from 100 MHz to 700 MHz; and charging circuitry configured to: convert the RF energy to a direct current (DC) power; and charge the rechargeable power supply with the DC power.

Example 2: The implantable medical device of example 1, wherein the antenna is configured to receive RF energy having one or more frequencies within the first range, and wherein the first range is from 12 MHz to 16 MHz.

Example 3: The implantable medical device of any of examples 1 and 2, wherein the antenna is configured to receive RF energy having one or more frequencies within the second range, and wherein the second range is from 200 MHz to 500 MHz.

Example 4: The implantable medical device of any of examples 1 through 3, wherein the antenna is configured to receive RF energy having a plurality of frequencies, and wherein the charging circuitry is configured to convert the RF energy at the plurality of frequencies to the DC power.

Example 5: The implantable medical device of any of examples 1 through 4, further comprising processing circuitry and communication circuitry, wherein the processing circuitry is configured to: determine a power level of the RF energy received by the antenna; and control the communication circuitry to transmit, to a charging device that generates the RF energy, an indication of the power level.

Example 6: The implantable medical device of any of examples 1 through 5, wherein the RF energy is first RF energy, and wherein the implantable medical device further comprises communication circuitry configured to determine communication information from a second RF energy received by the antenna.

Example 7: The implantable medical device of example 6, wherein the first RF energy and the second RF energy have a common frequency, and wherein the first RF energy is interleaved with the second RF energy.

Example 8: The implantable medical device of any of examples 6 and 7, wherein the first RF energy comprises a first frequency different than a second frequency of the second RF energy, and wherein the implantable medical device comprises a first bandpass filter configured to pass the first frequency of the first RF energy and a second bandpass filter configured to pass the second frequency of the second RF energy.

Example 9: The implantable medical device of any of examples 1 through 8, further comprising processing circuitry configured to: determine that the power source is charged to a predetermined threshold; and responsive to determining that the power source is charged to the predetermined threshold, controlling the charging circuitry to shunt the RF energy received from the antenna.

Example 10: The implantable medical device of any of examples 1 through 9, further comprising stimulation circuitry configured to generate electrical stimulation deliverable to a patient.

Example 11: A method comprising: receiving, via an antenna of an implantable medical device (IMD), radio frequency (RF) energy having one or more frequencies within at least one of a first range from 1 MHz to 20 MHz or a second range from 100 MHz to 700 MHz; converting, by charging circuitry of the IMD, the RF energy to a direct current (DC) power; and charging, by the charging circuitry of the IMD, a rechargeable power supply of the IMD with the DC power.

Example 12: The method of example 11, wherein receiving the RF energy comprises receiving the RF energy having one or more frequencies within the first range, and wherein the first range is from 12 MHz to 16 MHz.

Example 13: The method of any of examples 11 and 12, wherein receiving the RF energy comprises receiving the RF energy having one or more frequencies within the second range, and wherein the second range is from 200 MHz to 500 MHz.

Example 14: The method of any of examples 11 through 13, wherein receiving the RF energy comprises receiving the RF energy having a plurality of frequencies, and wherein converting the RF energy comprises converting the RF energy at the plurality of frequencies to the DC power.

Example 15: The method of any of examples 11 through 14, further comprising: determining a power level of the RF energy received by the antenna; and controlling communication circuitry to transmit, to a charging device that generates the RF energy, an indication of the power level.

Example 16: The method of any of examples 11 through 15, wherein the RF energy is first RF energy, and wherein the method further comprises determining communication information from a second RF energy received by the antenna.

Example 17: The method of example 16, wherein the first RF energy and the second RF energy have a common frequency, and wherein the first RF energy is interleaved with the second RF energy.

Example 18: The method of any of examples 16 and 17, wherein the first RF energy comprises a first frequency different than a second frequency of the second RF energy, and wherein the method further comprises: passing, via a first bandpass filter, the first frequency of the first RF energy; and passing, via a second bandpass filter, the second frequency of the second RF energy.

Example 19: The method of any of examples 11 through 18, further comprising: determining that the power source is charged to a predetermined threshold; and responsive to determining that the power source is charged to the predetermined threshold, controlling the charging circuitry to shunt the RF energy received from the antenna.

Example 20: The method of any of examples 11 through 19, further comprising generating, by stimulation circuitry, electrical stimulation deliverable to a patient.

Example 21: A system comprising: an external charging device comprising a first antenna configured to radiate radio frequency (RF) energy having one or more frequencies within at least one of a first range from 1 MHz to 20 MHz or a second range from 100 MHz to 700 MHz; and an implantable medical device (IMD) comprising: a second antenna configured to receive the RF energy; and charging circuitry configured to convert the RF energy to a direct current (DC) power and charge the rechargeable power supply with the DC power.

Example 22: The system of example 21, wherein the external charging device comprises an external programmer configured to program the IMD.

Example 23: The system of any of examples 21 and 22, wherein the RF energy comprises a first RF energy, and wherein the external charging device comprises a third antenna configured to radiate second RF energy having one or more frequencies within at least one of a first range from 1 MHz to 20 MHz or a second range from 100 MHz to 700 MHz, and wherein the second antenna is configured to receive the first RF energy radiated by the first antenna and the second RF energy radiated by the third antenna.

Example 24: The system of any of examples 21 through 23, wherein: the IMD comprises processing circuitry configured to: determine a power level of the RF energy received by the second antenna; and control communication circuitry to transmit, to the external charging device that generates the RF energy, an indication of the power level; and the external charging device comprises processing circuitry to: receive the indication of the power level; adjust, based on the indication of the power level, the one or more frequencies of the RF energy radiated by the first antenna.

Example 25: An implantable medical device comprising: a rechargeable power supply; an antenna configured to receive radio frequency (RF) energy; charging circuitry configured to: convert a first portion of the RF energy to a direct current (DC) power; and charge the rechargeable power supply with the DC power; and communication circuitry configured to: convert a second portion of the RF energy to a communication signal; and transmit the communication signal to processing circuitry.

Example 26: The implantable medical device of example 25, wherein the first portion of the RF energy is interleaved in time with the second portion of the RF energy, and wherein the implantable medical device further comprising processing circuitry configured to: determine a first period of time and a second period of time; control the charging circuitry to convert the first portion of the RF energy to the DC power during the first period of time; and control the communication circuitry to convert the second portion of the RF energy to the communication signal during the second period of time.

Example 27: The implantable medical device of any of examples 25 and 26, wherein the first portion of the RF energy comprises a first frequency of the RF energy and the second portion of the RF energy comprises a second frequency of the RF energy different than the first frequency; and wherein the implantable medical device comprises: a first bandpass filter configured to pass the first frequency of the first RF energy to the charging circuitry; and a second bandpass filter configured to pass the second frequency of the second RF energy to the communication circuitry.

Example 28: The implantable medical device of any of examples 25 through 27, wherein the RF energy comprises one or more frequencies within at least one of a first range from 1 MHz to 20 MHz or a second range from 100 MHz to 700 MHz.

Example 29: An external charging device comprising: an antenna configured to transmit RF energy to a direction; charging circuitry configured to apply an electrical signal to the antenna; and processing circuitry configured to: receive, via an implantable medical device (IMD), charging information indicative of RF energy received by the IMD; and adjust, based on the charging information, the direction in which the antenna transmits the RF energy.

Example 30: The external charging device of example 29, wherein the antenna comprises a phased array antenna comprising a plurality of antennae, and wherein the processing circuitry is configured to adjust the direction of RF energy transmission by adjusting, based on the charging information, a phase of one or more antennae of the plurality of antennae to adjust the direction in which the phased array antenna transmits the RF energy.

Example 31: The external charging device of any of examples 29 and 30, further comprising at least one motor configured to adjust a position of the antenna, and wherein the antenna comprises a directional antenna, and wherein the processing circuitry is configured to adjust the direction of RF energy transmission by controlling, based on the charging information, the at least one motor to adjust the direction of RF energy transmission.

Example 32: The external charging device of example 31, wherein the processing circuitry is configured to: control the at least one motor to sweep the directional antenna through a plurality of positions; control the charging circuitry to apply the electrical signal to the directional antenna at each position of the plurality of positions; receive, via the IMD, charging information indicative of the RF energy received by the IMD at each position of the plurality of positions; and control the at least one motor to adjust the position of the directional antenna by selecting one position of the plurality of positions for subsequent radiation of RF energy by the directional antenna.

Example 33: The external charging device of any of examples 29 through 32, wherein the processing circuitry is configured to select, based on the charging information indicative of the RF energy received by the IMD, a frequency of the RF energy to be radiated by the antenna.

Example 34: The external charging device of any of examples 29 through 33, wherein the processing circuitry is configured to: detect, based on the charging information, a reduction in power of the RF energy received by the IMD; and responsive to detect the reduction in power, adjust the direction of the antenna.

Example 35: The implantable medical device of any of examples 29 through 34, wherein the RF energy comprises one or more frequencies within at least one of a first range from 1 MHz to 20 MHz or a second range from 100 MHz to 700 MHz.

Example 36: An implantable medical device comprising: an antenna configured to receive radio frequency (RF) energy having one or more frequencies within at least one of a first range from 1 MHz to 20 MHz or a second range from 100 MHz to 700 MHz; and power circuitry configured to: convert the RF energy to a direct current (DC) power; and transfer the DC power for operation by the implantable medical device.

Example 37: The implantable medical device of example 36, further comprising a rechargeable power source, and wherein the power circuitry is configured to charge the rechargeable power source with the DC power.

Example 38: The implantable medical device of any of examples 36 and 37, further comprising a primary cell power source, and wherein the power circuitry is configured to transfer the DC power to operate at least a portion of the implantable medical device in addition to power from the primary cell power source.

Example 39: An implantable medical device comprising: a rechargeable power supply; a first antenna configured to receive first radio frequency (RF) energy at a frequency; charging circuitry configured to: convert at least a portion of the RF energy received by the first antenna to a direct current (DC) power; and charge the rechargeable power supply with the DC power; a second antenna configured to receive second RF energy at the frequency; and communication circuitry configured to: convert at least a portion of the RF energy received by the second antenna to a communication signal; and transmit the communication signal to processing circuitry.

The techniques described in this disclosure, including those attributed to system 10A, 10B, IMDs 14A, 14C, 14D, 21A, 21B, 338, 416 charging devices 20, 81, 101, 306, and programmer 19, and various constituent components, may be implemented, at least in part, in hardware, software, firmware or any combination thereof. For example, various aspects of the techniques may be implemented within one or more processors or processing circuitry, including one or more microprocessors, DSPs, ASICs, FPGAs, or any other equivalent integrated or discrete logic circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques or processes described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described herein. Any combination of the described operations or functions is contemplated. These and other examples are within the scope of the following claims.

What is claimed is:

1. An implantable medical device comprising:
a rechargeable power supply;
an antenna configured to receive radio frequency (RF) energy having one or more frequencies within at least one of a first range from 1 MHz to 20 MHz or a second range from 100 MHz to 700 MHz; and
charging circuitry configured to:
convert the RF energy to a direct current (DC) power; and
charge the rechargeable power supply with the DC power.

2. The implantable medical device of claim 1, wherein the antenna is configured to receive RF energy having one or more frequencies within the first range, and wherein the first range is from 12 MHz to 16 MHz.

3. The implantable medical device of claim 1, wherein the antenna is configured to receive RF energy having one or more frequencies within the second range, and wherein the second range is from 200 MHz to 500 MHz.

4. The implantable medical device of claim 1, wherein the antenna is configured to receive RF energy having a plurality of frequencies, and wherein the charging circuitry is configured to convert the RF energy at the plurality of frequencies to the DC power.

5. The implantable medical device of claim 1, further comprising processing circuitry and communication circuitry, wherein the processing circuitry is configured to:
determine a power level of the RF energy received by the antenna; and
control the communication circuitry to transmit, to a charging device that generates the RF energy, an indication of the power level.

6. The implantable medical device of claim 1, wherein the RF energy is first RF energy, and wherein the implantable medical device further comprises communication circuitry configured to determine communication information from a second RF energy received by the antenna.

7. The implantable medical device of claim 6, wherein the first RF energy and the second RF energy have a common frequency, and wherein the first RF energy is interleaved with the second RF energy.

8. The implantable medical device of claim 6, wherein the first RF energy comprises a first frequency different than a second frequency of the second RF energy, and wherein the implantable medical device comprises a first bandpass filter configured to pass the first frequency of the first RF energy and a second bandpass filter configured to pass the second frequency of the second RF energy.

9. The implantable medical device of claim 1, further comprising processing circuitry configured to:
determine that the power source is charged to a predetermined threshold; and
responsive to determining that the power source is charged to the predetermined threshold, controlling the charging circuitry to shunt the RF energy received from the antenna.

10. The implantable medical device of claim 1, further comprising stimulation circuitry configured to generate electrical stimulation deliverable to a patient.

11. A method comprising:
receiving, via an antenna of an implantable medical device (IMD), radio frequency (RF) energy having one or more frequencies within at least one of a first range from 1 MHz to 20 MHz or a second range from 100 MHz to 700 MHz;
converting, by charging circuitry of the IMD, the RF energy to a direct current (DC) power; and
charging, by the charging circuitry of the IMD, a rechargeable power supply of the IMD with the DC power.

12. The method of claim 11, wherein receiving the RF energy comprises receiving the RF energy having one or more frequencies within the first range, and wherein the first range is from 12 MHz to 16 MHz.

13. The method of claim 11, wherein receiving the RF energy comprises receiving the RF energy having one or more frequencies within the second range, and wherein the second range is from 200 MHz to 500 MHz.

14. The method of claim 11, wherein receiving the RF energy comprises receiving the RF energy having a plurality of frequencies, and wherein converting the RF energy comprises converting the RF energy at the plurality of frequencies to the DC power.

15. The method of claim 11, further comprising:
determining a power level of the RF energy received by the antenna; and
controlling communication circuitry to transmit, to a charging device that generates the RF energy, an indication of the power level.

16. The method of claim 11, wherein the RF energy is first RF energy, and wherein the method further comprises determining communication information from a second RF energy received by the antenna.

17. The method of claim 16, wherein the first RF energy and the second RF energy have a common frequency, and wherein the first RF energy is interleaved with the second RF energy.

18. The method of claim 16, wherein the first RF energy comprises a first frequency different than a second frequency of the second RF energy, and wherein the method further comprises:
passing, via a first bandpass filter, the first frequency of the first RF energy; and
passing, via a second bandpass filter, the second frequency of the second RF energy.

19. The method of claim 11, further comprising:
determining that the power source is charged to a predetermined threshold; and
responsive to determining that the power source is charged to the predetermined threshold, controlling the charging circuitry to shunt the RF energy received from the antenna.

20. The method of claim 11, further comprising generating, by stimulation circuitry, electrical stimulation deliverable to a patient.
